(19) United States
(12) Patent Application Publication
(10) Pub. No.: US 2009/0253768 A1
(43) Pub. Date: Oct. 8, 2009

(54) THERAPEUTIC COMPOUNDS FOR DISEASES AND DISORDERS

(75) Inventors: Christine Klein, Salt Lake City, UT (US); Andrew Gassman, Salt Lake City, UT (US); Leena Bhoite, Salt Lake City, UT (US); John Manfredi, Salt Lake City, UT (US)

Correspondence Address: Myriad PHARMACEUTICALS, Inc.;c/o CPA Global, P.O. Box 52050, Minneapolis, MN, 55402, US

(73) Assignee: Myriad Genetics, Incorporated, Salt Lake City, UT (US)

(21) Appl. No.: 12/400,580

(22) Filed: Mar. 9, 2009

Related U.S. Application Data
(63) Continuation of application No. PCT/US07/77888, filed on Sep. 7, 2007.
(60) Provisional application No. 60/842,777, filed on Sep. 7, 2006.

(51) Int. Cl. A61K 31/40; A61P 25/28

(52) U.S. Cl. 514/427

(57) ABSTRACT

Pyrrole derivatives are disclosed as agents for the treatment and prevention of neuropathies and neurodegenerative diseases characterized by the presence of axonal blockages, impaired axonal transport or impaired trafficking of vesicles in neurons.

### RELATED PRIORITY APPLICATIONS

This is a continuation of International Application PCT/US07/77888, with an international filing date of Sep. 7, 2007, now abandoned; which claims priority to U.S. Provisional Application Ser. No. 60/842,777 filed Sep. 7, 2006; both of which are incorporated by reference herein in their entireties.

### FIELD OF THE INVENTION

The invention provides a method for the therapeutic treatment of diseases and disorders associated with axonal transport defects or defects in the trafficking of vesicles and cellular components. In particular, the present invention is in the field of medicinal chemistry and relates to the use of specific derivatives of pyrroles for relieving axonal blockage and for the treatment of diseases and disorders associated with defects in axonal transport or intracellular vesicle trafficking.

### BACKGROUND OF THE INVENTION

Axonal transport, also called axoplasmic transport, is the process whereby cellular components, such as proteins, lipids, vesicles, organelles such as mitochondria, and messenger RNAs encoding proteins to be synthesized in the axonal processes (packaged as ribonucleoprotein particles, or RNPs), are actively transported between a neuron's cell body, or soma, and the cytoplasm of its axonal processes (axoplasm). Axons, which can be as much as 10,000 times longer than the width of a neuron's cell body, require these various cellular components for their function, growth, and general maintenance. Since many of these cellular components arise from processes that occur within the soma (e.g., transcription of nuclear genes), they must be transported from the cytoplasm of the soma to the axoplasm, where they are needed. Hence, axonal transport is critical for the growth, function and maintenance of axons and their synapses. Axonal transport is also used to move molecules that are to be degraded from the axoplasm to lysosomes within the soma, where they are broken down. Axonal transport is achieved through the action of specific molecular motors (kinesins, dynein, dynactin, etc.) and their associated adapter proteins, traveling along the cytoskeletal network of oriented microtubules, which extend through the cytoplasm from the cell body to the axoplasm. Movement of cellular components away from the cell body and towards the synapses at the ends of the axons, and towards the plus ends of the microtubules, is called anterograde transport. Movement of cellular components towards the cell body from the axoplasm, and towards the minus ends of the microtubules, is called retrograde transport.

Recent discoveries that mutations in specific microtubule motor proteins result in neurodegenerative diseases in humans have emphasized the importance of axonal transport to the vitality of neurons. Mutations compromising microtubule motor function have been found to cause hereditary forms of Charcot-Marie-Tooth disease (type 2A) (CMT2), hereditary spastic paraplegia and motor neuron disease, and the hallmarks of defective vesicular transport are observed in many other neurodegenerative diseases and disorders. See Holzbaur, Trends Cell Biol. 14:233-240 (2004); and Roy et al., Acta Neuropathol. (Berl.) 109:5-13 (2005). Additionally, neuropathies indicative of defective axonal transport have been documented in other classes of neurodegenerative disease. For example, focal bead-like swelling in dendrites and axons (neuritic beading) is seen in amyotrophic lateral sclerosis (ALS), (Delisle & Carpenter, J. Neurol. Sci. 63:241-250, 1984; Takahashi et al., Acta. Neuropathol. (Berl.) 94:294-299 (1997)) and Parkinson's disease (PD) (Mattila et al., Acta. Neuropathol. (Berl.) 98:157-164 (1999)). Indeed, genetic and histopathological evidence now suggests that impaired axonal transport may be involved in the pathoetiology of a variety of neurodegenerative diseases and disorders, including certain types of ALS, PD, CMT2, spinal muscular atrophy (SMA), and hereditary sensory motor neuropathy (HSMN). See Roy et al., Acta Neuropathol. (Berl.) 109:5-13 (2005).

In certain cases, impaired axonal transport has been shown to be the primary defect responsible for disease symptoms. For example, a subset of CMT2 patients have been shown to carry a loss-of-function mutation in the motor domain of a kinesin protein that participates in axonal transport of synaptic vesicle precursors (Zhao, et al., Cell. 105:587-597 (2001)). In other cases, the relationship between impaired axonal transport and the etiology of disease is less clear, but the suggestions are there. For example, although axonal growth defects contribute to the pathophysiology of SMA (see Jablonka, et al., J. Neurobiol. 58:272-286 (2004)), the role of axonal transport in the growth defects is unclear. However, given the importance of axonal transport for axonal growth, function and maintenance, axonal transport is a viable target for treatment of this disease as well.

The involvement of impaired vesicle trafficking within neurons in the pathophysiology of motor neuron diseases and disorders, as well as the similarities among the underlying biochemical lesions—were further illustrated by a recent study of an extended kindred by Nishimura and colleagues (Nishimura et al., Am. J. Hum. Genet. 75:822 (2004). They report that a single missense mutation in a vesicle trafficking protein, VAPB, results in three apparently distinct motor neuropathies: atypical ALS, typical severe ALS with rapid progression, and late-onset spinal muscular atrophy (LOSMA). Hence, therapies that specifically target this mutant protein may prove therapeutic for a number of apparently different diseases or disorders. More generally, however, compounds that enhance vesicle trafficking and axonal transport may be useful for a number of apparently disparate motor neuropathies.

Additionally, the outgrowth of neuronal processes, or neurites, and particularly axons, involved a great increase in the volume and surface area of the cell. This growth and expansion of neuronal processes is dependent upon the delivery of cellular components, such as proteins, lipids, vesicles, organelles, and RNPs from the soma to the site of growth. Consequently, growth cone advance is thought to be linked to the transport of the cytoskeletal and membrane constituents required for axonal growth. This linkage, particularly as pertains to the transport of membrane components, was demonstrated by the treatment of cultured hippocampal neurons with the antibiotic brefeldin A (BFA). Jareb and Banker, J. Neurosci. 17:8955-8963 (1197). BFA interferes with anterograde protein transport from the endoplasmic reticulum to the Golgi apparatus by inhibiting transport within the Golgi, which leads to proteins accumulating inside the ER. Treatment of the cultured hippcampal neurons with BFA resulted in the blockage of axonal transport, and the rapid inhibition of axonal growth. Jareb and Banker, J. Neurosci. 17:8955-8963 (1197). This linkage was subsequently confirmed in compartmented cultures of rat sympathetic neurons, wherein BFA treatment reversibly blocked axonal growth, as well as both anterograde transport of all proteins, and retrograde transport of at least nerve growth factor. Campenot et al., Neuropharmacol. 44:1107-1117 (2003). In view of this demonstrated linkage between axonal growth and axonal transport, it is reasonable to expect that defects in axonal transport can lead to reduced or diminished axonal growth, and, conversely, that stimulation of axonal transport, perhaps by relieving axonal blockages, can result in stimulated axonal growth.

The drugs currently used for treating the disorders and diseases listed above, and for promoting axonal growth, are only marginally efficacious and often have undesirable side effects. Thus, there is a large unmet need for better and safer drugs for the treatment or prevention, or for the delay of onset, or reversal, of symptoms of these diseases. Certainly there is a need for new classes of therapeutic compounds useful in treating such neuropathies, and providing relief to individuals suffering from these often tragic and debilitating disease and disorders.

### BRIEF SUMMARY OF THE INVENTION

The present invention is in the field of medicinal chemistry and relates to the use of pyrrole derivatives for the treatment and prophylaxis of disorders and diseases associated with impaired axonal transport or impaired vesicle trafficking within neurons.

In particular, pyrrole derivatives are disclosed as potential therapeutic agents for the treatment and prevention of diseases and disorders associated with impaired axonal transport or impaired vesicle trafficking within neurons include compounds of Formula I:

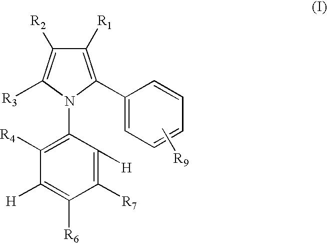

and pharmaceutically acceptable salts thereof, wherein:

R₁ is a hydrogen atom, halogen, hydroxy, alkyl, substituted alkyl, alkoxy, substituted alkoxy, or —CO₂R₁₀, and R₁₀ is alkyl or substituted alkyl;

R₂ is a hydrogen atom, halogen, hydroxy, alkyl, substituted alkyl, alkoxy, substituted alkoxy, or phenyl, optionally substituted with 0-5 phenyl substituents;

R₃ is a hydrogen atom, halogen, hydroxy, alkyl, substituted alkyl, alkoxy, or substituted alkoxy when R₂ is phenyl, or, when R₂ is not phenyl R₃ is —CH₂CH₂-phenyl optionally substituted with 0-5 phenyl substituents;

R₄ is a hydrogen atom, halogen, hydroxy, alkyl, substituted alkyl, alkoxy, or substituted alkoxy;

R₅ is a hydrogen atom, halogen, hydroxy, alkyl, substituted alkyl, alkoxy, or substituted alkoxy;

either of R₆ or R₇ is —(CH₂)ₙCO₂H or —O(CH₂)ₙCO₂H, wherein n is an integer from 0 to 4, or —(CH₂)ₘ—O—(CH₂)ₚCO₂H, wherein m is an integer from 1 to 2 and p is an integer from 1 to 2, while the other of R₆ or R₇ is a hydrogen atom, halogen, hydroxy, alkyl, substituted alkyl, alkoxy, or substituted alkoxy;

R₈ is a hydrogen atom, halogen, hydroxy, alkyl, substituted alkyl, alkoxy, or substituted alkoxy;

R₉ is 0-5 phenyl substituents, such as halogen (i.e., F, Cl, Br and I), hydroxy, or haloalkyl (such as trifluoromethyl).

The present invention also encompasses the use of the compounds of the invention for the preparation of pharmaceutical compositions that can be used for the treatment, prevention, or delay of onset of disorders and diseases associated with defects in axonal transport and vesicle trafficking in neurons, and can be used to promote axonal growth, in patients in need of such treatment. In particular, the compositions and methods of the present invention can be used to treat, prevent, or delay the onset of, or reverse the symptoms of, such diseases and disorders as ALS, PD, CMT2, SPA, SMA, HSMN, LOSMA, and other diseases and disorders involving impaired axonal transport or impaired vesicle trafficking within neurons, such as poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; hereditary spastic paraplegia; multiple sclerosis; Guillain-Barré syndrome; primary lateral sclerosis; taupathies including supranuclear palsy, corticobasal degeneration, Pick's disease, argyrophilic grain disease, and frontotemporal dementia and parkinsonism linked to chromosome 17; dementia with Lewy Bodies; Niemann-Pick type C disease; optic neuropathies; and diabetic neuropathy.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts the results of an experiment (described in Example 8), in which compound 16 (Tables 1 & 2) was used to suppress motor dysfunction in an animal model for amyotrophic lateral sclerosis.

### DETAILED DESCRIPTION OF THE INVENTION

It has been discovered by the inventors that certain pyrrole derivatives are capable of alleviating axonal transport defects in animal models of motor neuron diseases. These results suggest that certain pyrrole derivatives can be used to treat diseases and disorders associated with impaired axonal transport, or impaired vesicle trafficking in neurons, and can be used to promote axonal growth.

A mouse model of neurodegenerative disease was recently created in which axonal transport was impaired through a reduction of the dosage of kinesin-I (Stokin et al., Science 307:1282-1288 (2005)). Mice with 50% reduced levels of kinesin-I displayed a significant increase in axonal defects over matched controls, and the observed axonal defects were characterized as swellings that had accumulated abnormal amounts of microtubule-associated and motor proteins, organelles, and vesicles. These axonal defects, which are generically termed axonal blocks or axonal jams, are indicative of impaired anterograde axonal transport. The axonal blocks observed in the mice were similar to those reported in axons of strains of fruit flies (Drosophila melanogaster) that are mutant for vesicular transport (Micchelli et al., FASEB J. 17:79-81, 2003). In the case of the mutant Drosophila, such axonal blocks can result in a motor defect termed “tail-flipping”. That is, the mutant larvae exhibit loss of motor activity in the ventral posterior segments that causes an imbalance in body wall contractions; as a result, the larvae rhythmically flip their tails upward during locomotion. The Drosophila tail-flipping phenotype is considered to be a model for motor neuropathies such as amyotrophic lateral sclerosis (ALS; Lou Gehrig's disease).

In studies utilizing the Drosophila tail-flipping model for motor neuron dysfunction, the inventors discovered that the pyrrole derivatives of the present invention are capable of relieving the axonal blocks responsible for the tail-flipping phenotype. Hence, while not wishing to be bound by theory, the inventors believe that they have discovered a therapeutic approach to the treatment of neuropathies that are caused by impaired axonal transport, or impaired trafficking of vesicles within in neurons. Specifically inventors believe that they have discovered that certain pyrrole derivative can relieve axonal blocks, or alleviate defects in axonal transport, or defects in the transport of vesicles within neurons, and can be used to promote axonal growth.

Given that ALS, PD, CMT2, SMA, HSMN, LOSMA, and other diseases and disorders involve, or are caused by defects in axonal transport or impaired vesicle trafficking within neurons, the inventors believe that they have discovered that certain pyrrole derivatives can be used to treat, prevent, or delay the onset, or reverse the symptoms, of diseases and disorders such as ALS, PD, CMT2, SMA, HSMN, LOSMA, and any other diseases and disorders that involve, or are caused by, defects in axonal transport or impaired vesicle trafficking within neurons.

In addition to ALS, PD, CMT2, SMA, HSMN, and LOSMA, the inventors believe that the following motor neuron diseases and disorders might respond favorably to the methods of the present invention, at least in those cases where defects in axonal transport or impaired vesicle trafficking in neurons are involved:

PolyQ diseases: The expansion of CAG repeats encoding glutamine is known to cause several late-onset progressive neurodegenerative disorders, such as: Huntington disease, spinobulbar muscular atrophy, dentatorubral-pallidoluysian atrophy, Kennedy's disease (also called spinobulbar muscular atrophy [SBMA]), spinocerebellar ataxia 1, spinocerebellar ataxia 2, spinocerebellar ataxia 3, spinocerebellar ataxia 6, spinocerebellar ataxia 7, and spinocerebellar ataxia 17. These polyQ disorders commonly exhibit defects in axonal transport (Feany & La Spada, Neuron 40:1-2 (2003); Gunawardena et al., Neuron 40:25-40 (2003); Szebenyi et al., Neuron 40:41-52 (2003)). Indeed, evidence suggests that perturbations in axonal transport pathways are an early event in polyQ disease (Gunawardena & Goldstein, Arch Neurol. 62:46 (2005)).

Traumatic brain and spinal cord injury: Traumatic brain injury (TBI) is marked by rapid and long-term accumulation of proteins in and around axonal processes within the brain. TBI is also an epigenetic risk factor for developing neurodegenerative disorders, such as AD and PD (Smith et al., Neuromolecular Med. 4:59-72 (2003)). The ability of certain pyrrole derivatives to relieve axonal blockages in animal models suggests their possible use in treating traumatic injury to both the brain and spinal column.

Hereditary spastic paraplegia (HSP): These motor neuron diseases exhibit clear cytoskeletal abnormalities that suggest the involvement of impaired axonal transport in the pathogenesis of the diseases (Coleman and Perry, Trends Neurosci. 25:532-537 (2002)).

Multiple sclerosis (MS): Inflammation is the cause of much neural damage in multiple sclerosis, resulting in impaired axonal transport (Neumann, Curr. Opin. Neurol. 16:267-273 (2003)). These observations admit the possibility that the neurodegeneration experienced by MS patients may be attenuated by agents that enhance axonal transport. In a similar vein, diseases such as Guillain-Barré syndrome, an inflammatory disorder of the peripheral nerves, may be amenable to therapeutic intervention with agents that enhance axonal transport.

Miscellaneous motor neuron disorders: Primary lateral sclerosis (PLS) is a rare degenerative disorder of the upper motor neuron, whose classification is controversial (Swash et al., J. Neurol. Sci. 170:5-10 (1999)). In fact, a recent study has concluded that PLS is not a discrete nosological entity but represents one end of a continuous spectrum of motor neuron disease (LeForestier et al., Brain 124 (Pt. 10):1989-1999 (2001)). A therapeutic agent that successfully treats one type of motor neuron dysfunction is therefore a candidate for treatment of other motor neuron disorders.

Tauopathies: Aberrant functions of the microtubule-associated proteins collectively called tau can lead to neurodegenerative disorders like progressive supranuclear palsy, corticobasal degeneration, Pick's disease, argyrophilic grain disease, and frontotemporal dementia and parkinsonism linked to chromosome 17 (FTDP-17) (Goedert & Jakes. Biochim. Biophys. Acta 1739:240-250 (2005); Buée et al. Brain Res. Brain Res. Rev. 33:95-130 (2000)). One feature of tauopathies is the clear disruption of axonal transport that accompanies them.

Dementia with Lewy Bodies (DLB): DLB is characterized by the presence of cytoplasmic inclusions of alpha-synuclein in the cerebral cortex and in the nuclei of the brain stem (Rampello et al., Arch. Gerontol. Geriatr. 39:1-14 (2004)). Such protein aggregates apparently disrupt vesicle transport in proximal neurons. A therapy that treats dysfunctional vesicle transport is a candidate for the treatment of DLB.

Niemann-Pick type C disease (NPC): The primary lesion of NPC appears to be impaired cholesterol trafficking and excessive glycosphingolipid storage. One consequence of this impairment is abnormal vesicle trafficking in neural tissue, which likely contributes to the neurodegeneration characteristic of the disease (Nixon, Neurobiol. Aging 26:373-382 (2005)). A recent study indicates that the abnormal vesicle trafficking may contribute to the neurodegeneration seen in the brain tissue of NPC patients (Jin et al., Am. J. Pathol. 164:975-985 (2004)). Accordingly, compounds that enhance vesicle trafficking in neurons may treat, or relieve the symptoms, of NPC.

Optic neuropathies: Histological evidence suggests impaired axonal transport of mitochondria in Leber's hereditary optic neuropathy (LHON) and in Cuban epidemic of optic neuropathy (CEON). Since mitochondria are transported along microtubules by mechanisms similar to microtubule-directed transport of vesicles, the pyrrole derivatives of the present invention could potentially be used to treat these diseases, or reduce or reverse their symptoms.

Diabetic neuropathy (DN): In addition to the involvement of impaired axonal transport, or impaired vesicle trafficking, in the pathoetiology of the neurodegenerative diseases and disorders outlined above, diabetic neuropathy is also characterized by impaired axonal transport. (See McLean, Neurochem Res. 22:951-956 (1997) and Schoemaker, Diabetes Care. 17:1362 (1994).) In certain rodent models of diabetes expression deficits occur in nerve growth factor (NGF), and in its high-affinity receptor, trkA. These expression deficits lead to decreased retrograde axonal transport of NGF, decreased support of NGF-dependent sensory neurons, and reduced expression of neuropeptides, substance P, and calcitonin gene-related peptide (CGRP). (Tomlinson, et al., Philos. Trans. R. Soc. Lond. B. Biol. Sci. 351:455-462 (1996)) Hence, compounds that enhance vesicle trafficking in neurons, such as the pyrrole derivatives of the instant invention, may also be useful for the treatment or prophylaxis of DN.

The possibility of treating DN with the methods of the instant invention is particularly important because the disorder is highly prevalent among diabetes patients, and the total annual cost of DN and its complications in the U.S. alone was estimated in 2003 to be between 4.6 and 13.7 billion U.S. dollars (Gordois et al., Diabetes Care 26:1790-1795 (2003)). Furthermore, up to 27% of the direct medical costs associated with the care of diabetes patients can be attributed to the treatment of DN and its associated symptoms (Gordois et al., Diabetes Care 26:1790-1795 (2003)).

Importantly, the present invention provides specific pyrrole derivatives that, when tested in a Drosophila model for ALS, relieved axonal blockages that are characteristic of impaired axonal transport and impaired vesicle trafficking in neurons. Consequently the pyrrole derivatives of the present invention have therapeutic potential in the treatment or prophylaxis those diseases and disorders involving impaired axonal transport or impaired vesicle trafficking in neurons that are characterized by the presence of such axonal blocks, and in promoting axonal growth. In particular, the present invention provides specific pyrrole derivatives that can be used to treat, prevent, or delay the onset of symptoms, or reverse the symptoms of ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; and DN.

The present invention also provides pharmaceutical compositions comprising the therapeutic compounds of the present invention and a pharmaceutically acceptable excipient or carrier for use in treating the diseases and disorders mentioned above. Such pharmaceutical compositions are formulated in order to deliver a therapeutically effective, or prophylactically effective, amount of the compound to a patient in need of such treatment.

The present invention also provides therapeutic methods that make use of the therapeutic compounds and compositions of the present invention for the treatment of treatment, prevention, or delay of onset or reversal of symptoms of neuropathies and other diseases and disorders that are caused by, or otherwise involve impairment of axonal transport or impairment of vesicle transport in neurons, or are characterized by the presence of axonal blocks. In particular, the therapeutic methods of the present application can be used to treat, prevent, or delay the onset of or reverse the symptoms of such diseases and disorders, including, but not specifically limited to, ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; and DN, in patients in need of such treatment.

The present invention and various embodiments thereof are described in more detail following these definitions.

### DEFINITIONS

As used herein, the terms pertaining to the compounds of the invention have the meanings set forth below.

“Alkyl” is a straight chain or branched, cyclic or noncyclic, saturated or unsaturated alkyl containing from 1 to 12 carbon atoms (also referred to herein as “C₁₋₁₂ alkyl”). Similarly, a “lower alkyl” is as defined above, but contains from 1 to 4 carbon atoms (also referred to herein as a “C₁₋₄ alkyl”). Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like. Representative unsaturated cyclic alkyls include cyclopentenyl and cyclohexenyl, and the like. Alkyls include “alkoxy” as defined below.

“Alkoxy” is an alkyl having at least one alkyl hydrogen atom replaced with an oxygen atom, such as methoxy, ethoxy, n-propoxy, n-butoxy, n-pentoxy, isopropoxy, sec-butoxy and the like. “Lower alkoxy” has same meaning, but utilizing lower alkyl in place of alkyl.

“Aminoalkyl” is a straight chain or branched, cyclic or noncyclic, saturated or unsaturated alkyl containing from 1 to 12 carbon atoms with at least one alkyl hydrogen atom or carbon atom replaced with —NH₂ or —NH—, respectively (also referred to herein as “C₁₋₁₂ aminoalkyl”).

“Aryl” is an aromatic carbocyclic moiety contain from 6 to 12 carbon atoms (also referred to herein as a “C₆₋₁₂ aryl”), such as phenyl and naphthyl. Aryls include aryloxy, as defined below.

“Aryloxy” is an aryl having at least one aryl hydrogen atom replaced with an oxygen atom, such as phenoxy and the like.

“Arylalkyl” is an alkyl having at least one alkyl hydrogen atom replaced with an aryl moiety, such as benzyl, —(CH₂)₂phenyl, —(CH₂)₃phenyl, —CH(phenyl)₂, and the like. Arylalkyls include arylalkoxy as defined below.

“Arylalkoxy” is an arylalkyl having at least one alkyl hydrogen replaced with an oxygen atom, such as benzoxy and the like. “Alkylaryloxy” is an arylalkyl having at least one aryl hydrogen replaced with an oxygen atom, such as hydroxy benzyl and the like.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Thus, in addition to the heteroaryls listed below, heterocycles also include morpholinyl, pyrrolidinonyl, pyrrolidinyl, piperidinyl, hydantoinyl, valerolactamyl, oxiranyl, oxetanyl, aziridinyl, azetidinyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl and the like.

“Heterocyclealkyl” means an alkyl having at least one alkyl hydrogen atom replaced with a heterocycle moiety, such as —CH₂(heterocycle), —(CH₂)₂(heterocycle) and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl and the like.

“Heteroarylalkyl” means an alkyl having at least one alkyl hydrogen atom replaced with a heteroaryl moiety, such as —CH₂pyridinyl, —CH₂pyrimidinyl, and the like.

The term “substituted” as used herein means any of the above groups—that is, alkyl, aryl, arylalkyl, heterocycle, heterocyclealkyl, heteroaryl or heteroarylalkyl—wherein at least one hydrogen atom is replaced with a substituent. In the case of an oxo substituent (“═O”), two hydrogen atoms are replaced. A “substituent” in this regard is halogen (such as F, Cl, Br and I), oxo, hydroxy, haloalkyl (such as trifluoromethyl), —R, —OR, —C(═O)R, —C(═O)OR, —C(═O)NRR, —NRR, —NRC(═O)R, —NRC(═O)OR, —NRC(═O)NRR, —OC(═O)R, —OC(═O)OR, —OC(═O)NRR, —SH, —SR, —SOR, —SO₂R, —SO₂NR₂, —NRSO₂R, —NR₂SO₂R, —Si(R)₃, or —OP(OR)₃, wherein each occurrence of R is the same or different and independently hydrogen, alkyl, substituted alkyl, aryl, substituted aryl, arylalkyl, substituted arylalkyl, heterocycle, substituted heterocycle, heterocyclealkyl or substituted heterocyclealkyl, or wherein any two R groups attached to the same nitrogen atom, taken together with the nitrogen atom to which they are attached, form a heterocyclic ring or a substituted heterocyclic ring.

The term “phenyl substituent” has the same meaning as defined above for “substituent,” except that it does not include an oxo substituent. In specific embodiments, phenyl substituents are halogen, hydroxy or haloalkyl.

As used herein, the term “preventing,” when used in the context of “preventing a disease or disorder,” refers both to not allowing a symptom to increase or worsen, as well as to reducing or slowing the rate of increase or worsening of the symptoms of the disease or disorder. For example, a symptom that can be measured could be the sensory sensitivity or fine motor control of an extremity of a patient. Preventing an increase, according to the definition provided herein, means that the amount of the symptom (e.g., sensory sensitivity loss or fine motor control decline) does not increase or worsen, or that the rate at which it increases or worsens is reduced.

As used herein, the term “treating a disease or disorder,” “treating a neuropathy,” or “treating a disease or disorder associated with impaired axonal transport or impaired vesicle trafficking” refers to a slowing of the progression of the disease or disorder, or its symptoms, or a reversal of the disease or disorder, or its symptoms. For example, “treating ALS” includes not only treating a disease, but reducing or reversing a symptom or symptoms of that disease.

As used herein, the term “preventing a disease or disorder,” or “preventing a neuropathy,” or “preventing a disease or disorder associated with impaired axonal transport or impaired vesicle trafficking” refers to a slowing of the disease progression or slowing or stopping of the onset of the disease or the symptoms thereof. For example, “preventing ALS” can include stopping the onset of ALS or the symptoms thereof, or reversing the symptoms of ALS once they are manifest.

As noted above, the present invention provides methods for treating, or preventing, or delaying the onset of, or reversing the symptoms of diseases and disorders involving impairment of axonal transport, or the impairment of vesicle trafficking in neurons, specifically the axons of neurons. Additionally the present invention provides methods for treating, or preventing, or delaying the onset of, or reversing the symptoms of diseases and disorders characterized by the presence of axonal blocks or blockages, or axonal jams. These methods can be applied in any such neurodegenerative disease or disorder, but have clear application in ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; and DN. Such, methods have in common the alleviation of axonal blocks, or alleviating impaired axonal transport or impaired vesicle trafficking in axons. While not wishing to be bound by theory, it is believed that by alleviating axonal blocks, alleviating impaired axonal transport or alleviating impaired vesicle trafficking in axons in an individual, through the administration of an effective amount of a compound or composition described herein, the diseases and disorders listed above can be treated or prevented, or the symptoms of such diseases can be alleviated, slowed, reversed, or even eliminated.

Generally, the invention relates to the concept that compounds of Formulae I-VI can be used to alleviate of axonal blocks, or alleviate impaired axonal transport, or alleviate impaired vesicle trafficking in axons. Thus, diseases characterized by the presence of axonal blocks, or involving impaired axonal transport, or impaired vesicle trafficking in axons, can be treated or prevented with the methods of the invention, which are specifically designed to alleviate such blocks or impairments in a patient.

Importantly, however, the methods in the present invention may also be used prophylactically in patients at risk of developing neurodegenerative diseases and disorders characterized by the presence of axonal blocks, or by impaired axonal transport, or impaired vesicle trafficking in axons. Such patients may be identified by any acceptable method in the art, such as through genotyping by any suitable method, or by analysis of their family's history of disease, or through pedigree analysis, or through characterization of symptoms. Methods of determining the genotype of an individual include nucleic acid sequencing, selective hybridization, allele-specific amplification, and the like. For patients found to be at risk by such methods, the methods of the present invention may be used to prevent or delay the onset of symptoms of the diseases and disorders involved; including such diseases and disorders as ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; and DN.

### Therapeutic Compounds

The present invention includes compounds of Formula I:

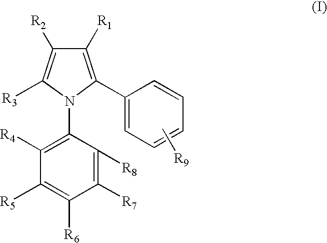

or pharmaceutically acceptable salts thereof, wherein:

R₁ is a hydrogen atom, halogen, hydroxy, alkyl, substituted alkyl, alkoxy, substituted alkoxy, or —CO₂R₁₀, and R₁₀ is alkyl or substituted alkyl;

R₂ is a hydrogen atom, halogen, hydroxy, alkyl, substituted alkyl, alkoxy, substituted alkoxy, or phenyl, optionally substituted with 0-5 phenyl substituents;

R₃ is a hydrogen atom, halogen, hydroxy, alkyl, substituted alkyl, alkoxy, or substituted alkoxy when R₂ is phenyl, or, when R₂ is not phenyl R₃ is —CH₂CH₂-phenyl optionally substituted with 0-5 phenyl substituents;

R₄ is a hydrogen atom, halogen, hydroxy, alkyl, substituted alkyl, alkoxy, or substituted alkoxy;

R₅ is a hydrogen atom, halogen, hydroxy, alkyl, substituted alkyl, alkoxy, or substituted alkoxy;

either of R₆ or R₇ is —(CH₂)ₙCO₂H or —O(CH₂)ₙCO₂H, wherein n is an integer from 0 to 4, or —(CH₂)ₘ—O—(CH₂)ₚCO₂H, wherein m is an integer from 1 to 2 and p is an integer from 1 to 2, while the other of R₆ or R₇ is a hydrogen atom, halogen, hydroxy, alkyl, substituted alkyl, alkoxy, or substituted alkoxy;

R₈ is a hydrogen atom, halogen, hydroxy, alkyl, substituted alkyl, alkoxy, or substituted alkoxy; and

R₉ is 0-5 phenyl substituents, such as halogen (e.g., F, Cl, Br or I), hydroxy, or haloalkyl (e.g., trifluoromethyl).

In one set of embodiments, R₁, R₅ and R₈ are hydrogen atoms, and the compounds, and pharmaceutically acceptable salts thereof, are in accordance with Formula II:

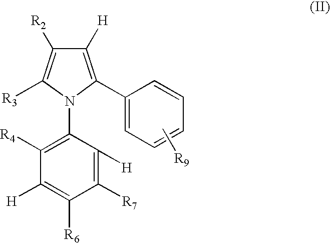

wherein R₂, R₃, R₄, R₆, R₇, and R₉ are as defined above.

In another set of embodiments of the present invention R₂ is phenyl and R₃ is either a hydrogen atom, halogen, hydroxy, alkyl, substituted alkyl, alkoxy, or substituted alkoxy, such that, in this set of embodiments, the compounds, and pharmaceutically acceptable salts thereof, correspond to Formula III:

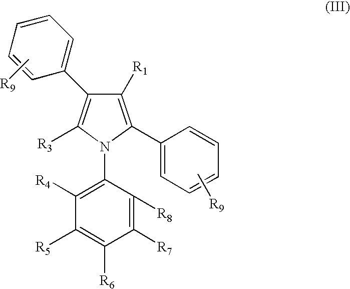

wherein R₁, R₄, R₅, R₆, R₇, R₈ and R₉ are as defined above.

In a subset of these embodiments, R₁, R₅ and R₈ are hydrogen atoms, and the compounds, and pharmaceutically acceptable salts thereof, are in accordance with Formula IV:

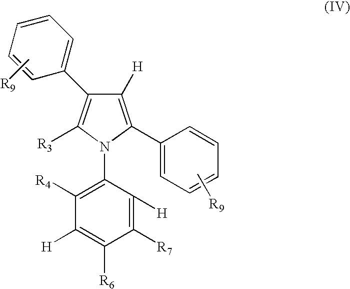

wherein R₃, R₄, R₆, R₇, and R₉ are as defined above.

In another set of embodiments of the present invention R₂ is either a hydrogen atom, halogen, hydroxy, alkyl, substituted alkyl, alkoxy, or substituted alkoxy, and R₃ is —CH₂CH₂-phenyl, such that, in this set of embodiments, the compounds, and pharmaceutically acceptable salts thereof, correspond to Formula V:

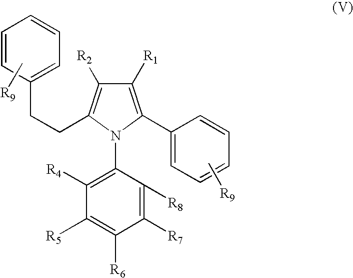

wherein R₁, R₄, R₅, R₆, R₇, R₈ and R₉ are as defined above.

In a subset of these embodiments, R₁, R₅ and R₈ are hydrogen atoms, and the compounds, and pharmaceutically acceptable salts thereof, are in accordance with Formula VI:

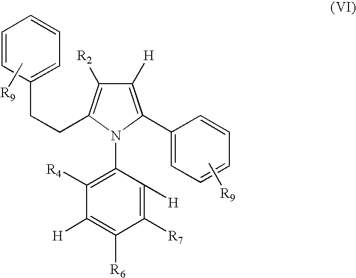

wherein R₂, R₄, R₆, R₇ and R₉ are as defined above.

It should be noted that in most embodiments of the present invention, one of either of R₆ or R₇ is —(CH₂)ₙCO₂H, or —O(CH₂)ₙCO₂H, wherein n is an integer from 0 to 4, or —(CH₂)ₘ—O—(CH₂)ₚCO₂H, wherein m is an integer from 1 to 2 and p is an integer from 1 to 2, while the other of R₆ or R₇ is a hydrogen atom, halogen, hydroxy, alkyl, substituted alkyl, alkoxy, or substituted alkoxy. While not wishing to be bound by theory, the presence of a carboxyl group, in the form of a carboxylic acid substituent at one of these two positions (R₆ or R₇) may be important for the efficacy of the compound in inhibiting Aβ₄₂ secretion. As indicated, in certain embodiments of the present invention, the carboxylic acid substituent is linked directly to the aromatic ring at either R₆ or R₇. In other embodiments, the carboxylic acid substituent is linked through an ether linkage to the aromatic ring at either R₆ or R₇. In either case, the carboxylic acid group can be appended to either the R₆ or the R₇ position, although substitution at the R₇ position may be preferred.

It should also be noted that the carboxylic acid group appended to either the R₆ or the R₇ position can potentially be created by the hydrolytic cleavage of an ester. Consequently, in certain embodiments, the compounds of the present invention further include such esters of all compounds according to Formulae I-VI. (See, for example Compound #27 in Table 1, below.) Such esters can include methyl esters and ethyl esters, as well as other lower alkyl esters.

In still other embodiments of the present invention, in all compounds according to Formulae I-VI, one of either R₆ or R₇ is substituted with a bioisostere of carboxylic acid, including: -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, L-C(═O)CH₂SH, -L-C(═O)NHCN, -L-sulfo, -L-(2,6 difluorophenol), L-phosphono, and -L-tetrazolyl;

wherein L can be saturated, partially saturated, or unsaturated, and is selected from the group consisting of —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, wherein each n is an integer independently selected from 0, 1, 2, 3, 4, 5, 6, 7, and 8, wherein each carbon can be optionally substituted with one or more C₁₋₃ alkyl or C₃₋₆ cycloalkyl. However, the compounds of the present invention specifically exclude the compound 4-[2-(4-Fluoro-phenyl)-4-phenyl-pyrrol-1-yl]-benzenesulfonamide (CAS REGISTRY No. 197904-68-0).

In certain embodiments, the present invention provides the specific compounds identified in Table 1, below. In all cases, the compounds of the present invention are those that are effective at alleviating axonal blocks, or alleviating impairments in axonal transport or vesicle trafficking in neurons.

TABLE 1
Example Compounds

| Compound | | | |
|---|---|---|---|
| No. | Compound Structure | MW | Compound Name |
| 1 | 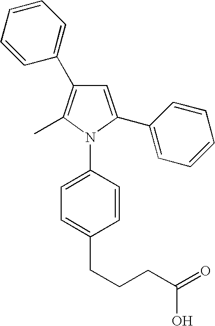 | 395.50 | 4-[4-(2-Methyl-3,5-diphenyl- pyrrol-1-yl)-phenyl]-butyric acid |
| 2 | 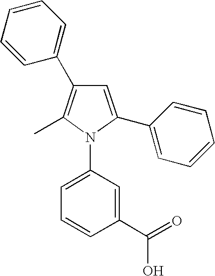 | 353.42 | 3-(2-Methyl-3,5-diphenyl- pyrrol-1-yl)-benzoic acid |
| 3 | 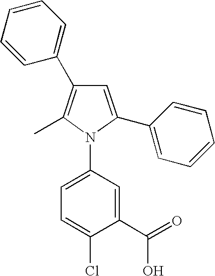 | 387.86 | 2-Chloro-5-(2-methyl-3,5- diphenyl-pyrrol-1-yl)- benzoic acid |
| 4 | 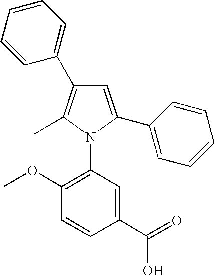 | 383.44 | 4-Methoxy-3-(2-methyl-3,5- diphenyl-pyrrol-1-yl)- benzoic acid |
| 5 | 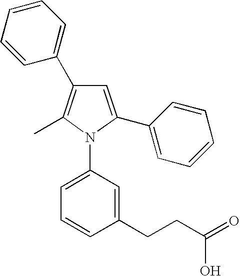 | 381.47 | 3-[3-(2-Methyl-3,5-diphenyl- pyrrol-1-yl)-phenyl]- propionic acid |
| 6 | 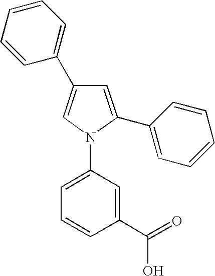 | 339.39 | 3-(2,4-Diphenyl-pyrrol-1-yl)- benzoic acid |
| 7 | 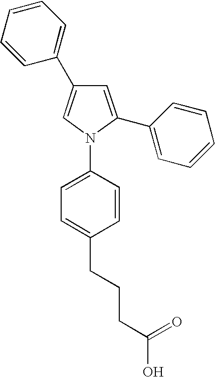 | 381.47 | 4-[4-(2,4-Diphenyl-pyrrol-1- yl)-phenyl]-butyric acid |
| 8 | 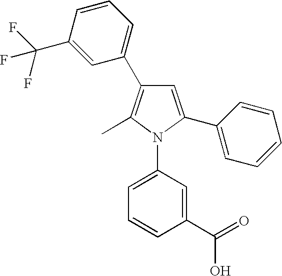 | 421.42 | 3-[2-Methyl-5-phenyl-3-(3- trifluoromethyl-phenyl)-pyrrol- 1-yl]-benzoic acid |
| 9 | 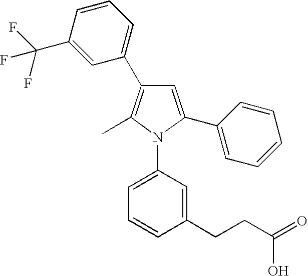 | 449.47 | 3-{3-[2-Methyl-5-phenyl-3-(3- trifluoromethyl-phenyl)-pyrrol- 1-yl]-phenyl}-propionic acid |
| 10 | 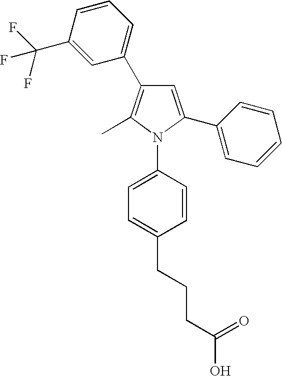 | 463.50 | 4-{4-[2-Methyl-5-phenyl-3-(3- trifluoromethyl-phenyl)-pyrrol- 1-yl]-phenyl}-butyric acid |
| 11 | 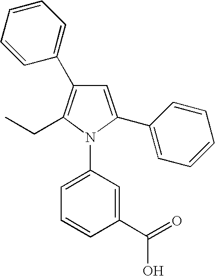 | 367.45 | 3-(2-Ethyl-3,5-diphenyl- pyrrol-1-yl)-benzoic acid |
| 12 | 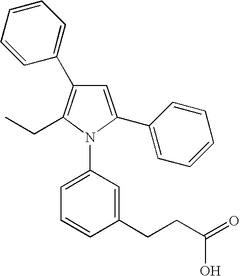 | 395.50 | 3-[3-(2-Ethyl-3,5-diphenyl- pyrrol-1-yl)-phenyl]- propionic acid |
| 13 | 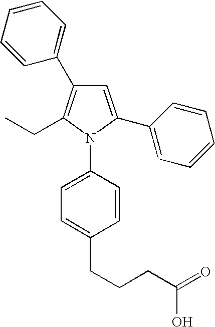 | 409.53 | 4-[4-(2-Ethyl-3,5-diphenyl- pyrrol-1-yl)-phenyl]- butyric acid |
| 14 | 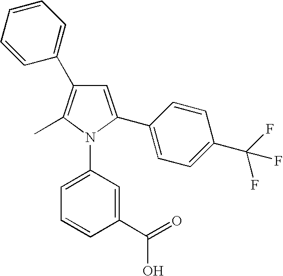 | 421.42 | 3-[2-Methyl-3-phenyl-5-(4- trifluoromethyl-phenyl)-pyrrol- 1-yl]-benzoic acid |
| 15 | 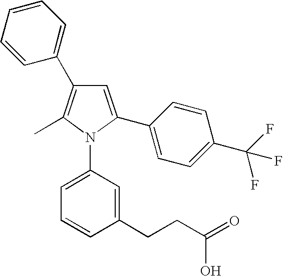 | 449.47 | 3-{3-[2-Methyl-3-phenyl-5-(4- trifluoromethyl-phenyl)-pyrrol- 1-yl]-phenyl}-propionic acid |
| 16 | 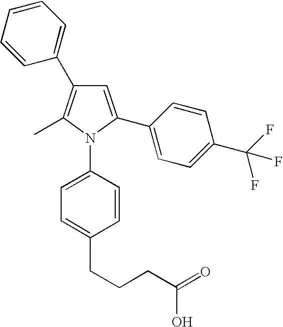 | 463.50 | 4-{4-[2-Methyl-3-phenyl-5-(4- trifluoromethyl-phenyl)-pyrrol- 1-yl]-phenyl}-butyric acid |
| 17 | 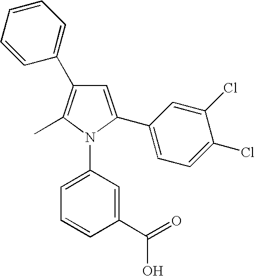 | 422.31 | 3-[5-(3,4-Dichloro-phenyl)-2- methyl-3-phenyl-pyrrol-1-yl]- benzoic acid |
| 18 | 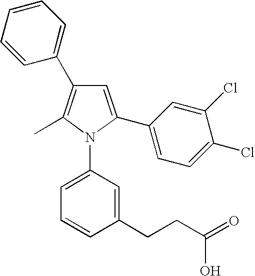 | 450.36 | 3-{3-[5-(3,4-Dichloro-phenyl)- 2-methyl-3-phenyl-pyrrol-1- yl]-phenyl}-propionic acid |
| 19 | 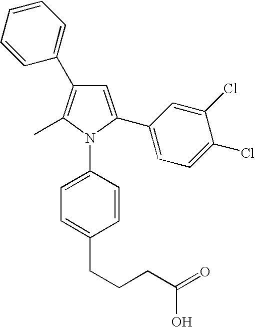 | 464.39 | 4-{4-[5-(3,4-Dichloro-phenyl)- 2-methyl-3-phenyl-pyrrol-1- yl]-phenyl}-butyric acid |
| 20 | 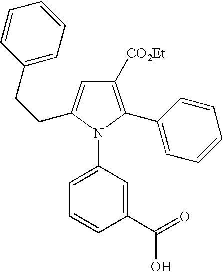 | 439.51 | 1-(3-Carboxy-phenyl)-5- phenethyl-2-phenyl-1H- pyrrole-3-carboxylic acid ethyl ester |
| 21 | 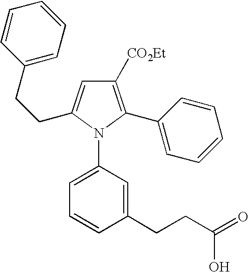 | 467.56 | 1-[3-(2-Carboxy-ethyl)- phenyl]-5-phenethyl-2- phenyl-1H-pyrrole-3- carboxylic acid ethyl ester |
| 22 | 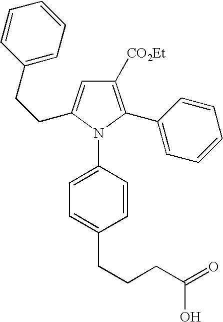 | 481.59 | 1-[4-(3-Carboxy-propyl)- phenyl]-5-phenethyl-2- phenyl-1H-pyrrole-3- carboxylic acid ethyl ester |
| 23 | 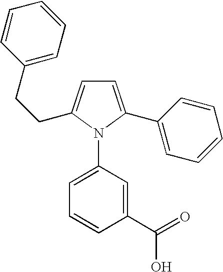 | 367.45 | 3-(2-Phenethyl-5-phenyl- pyrrol-1-yl)-benzoic acid |
| 24 | 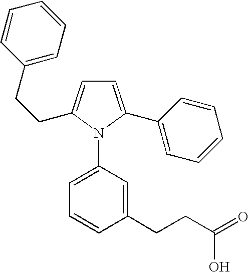 | 395.50 | 3-[3-(2-Phenethyl-5-phenyl- pyrrol-1-yl)-phenyl]- propionic acid |
| 25 | 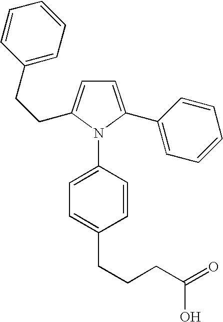 | 409.53 | 4-[4-(2-Phenethyl-5-phenyl- pyrrol-1-yl)-phenyl]- butyric acid |
| 26 | 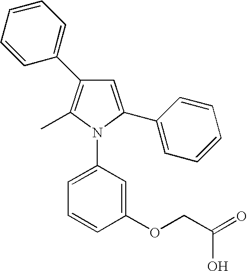 | 383.44 | [3-(2-Methyl-3,5-diphenyl- pyrrol-1-yl)-phenoxy]- acetic acid |
| 27 | 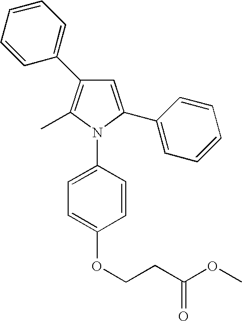 | 411.50 | 3-[4-(2-Methyl-3,5-diphenyl- pyrrol-1-yl)-phenoxy]- propionic acid methyl ester |
| 28 | 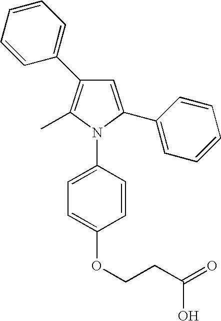 | 397.47 | 3-[4-(2-Methyl-3,5-diphenyl- pyrrol-1-yl)-phenoxy]- propionic acid |
| 29 | 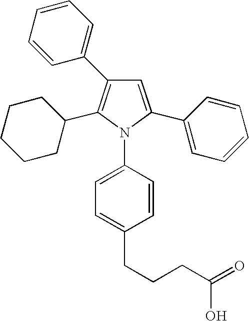 | 463.62 | 4-[4-(2-Cyclohexyl-3,5- diphenyl-pyrrol-1-yl)-phenyl]- butyric acid |
| 30 | 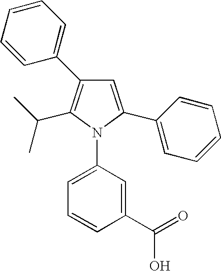 | 381.47 | 3-(2-Isopropyl-3,5-diphenyl- pyrrol-1-yl)-benzoic acid |
| 31 | 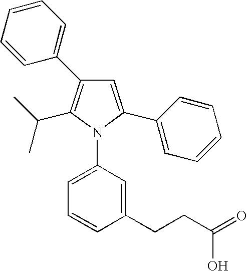 | 409.53 | 3-[3-(2-Isopropyl-3,5- diphenyl-pyrrol-1-yl)-phenyl]- propionic acid |
| 32 | 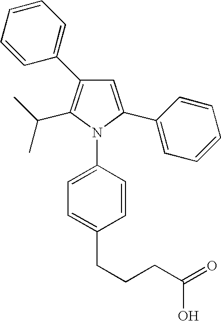 | 423.55 | 4-[4-(2-Isopropyl-3,5- diphenyl-pyrrol-1-yl)-phenyl]- butyric acid |
| 33 | 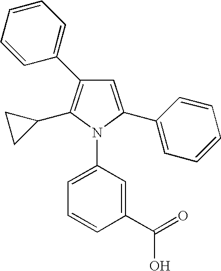 | 379.46 | 3-(2-Cyclopropyl-3,5- diphenyl-pyrrol-1-yl)- benzoic acid |
| 34 | 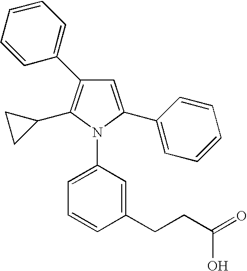 | 407.51 | 3-[3-(2-Cyclopropyl-3,5- diphenyl-pyrrol-1-yl)-phenyl]- propionic acid |
| 35 | 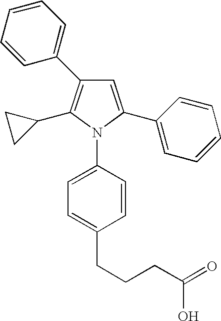 | 421.54 | 4-[4-(2-Cyclopropyl-3,5- diphenyl-pyrrol-1-yl)-phenyl]- butyric acid |
| 36 | 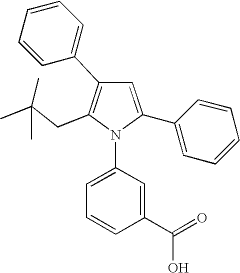 | 409.53 | 3-[2-(2,2-Dimethyl-propyl)- 3,5-diphenyl-pyrrol-1-yl]- benzoic acid |
| 37 | 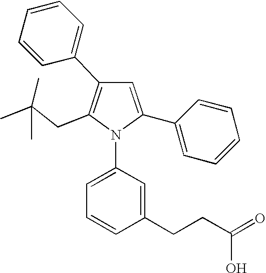 | 437.58 | 3-{3-[2-(2,2-Dimethyl-propyl)- 3,5-diphenyl-pyrrol-1-yl]- phenyl}-propionic acid |
| 38 | 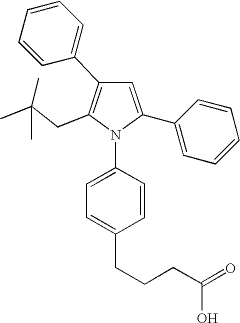 | 451.61 | 4-{4-[2-(2,2-Dimethyl-propyl)- 3,5-diphenyl-pyrrol-1-yl]- phenyl}-butyric acid |
| 39 | 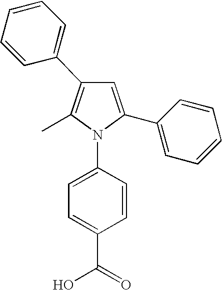 | 353.42 | 4-(2-Methyl-3,5-diphenyl- pyrrol-1-yl)-benzoic acid |

Without wishing to be bound by theory, it is believed that the pyrrole derivatives of the present invention can alleviate axonal blocks or alleviate impaired axonal transport or impaired vesicle trafficking within neurons. In so doing, it is believed that the compounds of the present invention are useful for treating and/or preventing diseases and disorders characterized by the presence of neuronal blocks, or involving impaired axonal transport or impaired vesicle trafficking within neurons. It is also believed that compounds of the present invention can be used to promote axonal growth, by alleviating axonal transport blockages or axonal transport defects. Thus, in one aspect of this invention, which is described in detail below, methods of treating such diseases are provided comprising identifying a patient in need of such treatment, and administering to that patient an effective amount of a pyrrole derivative of the present invention. Preferably, the pyrrole derivative that is used in the methods of the invention is capable of affecting a reduction of symptoms of such diseases by at least 10, 20, 30, 40, or 50 percent, at a concentration of 10 μM.

Preferred pyrrole derivatives for use in the methods of the invention are those that have an IC50 in assays of tail-flipping in Drosophila (such as the assays described in Examples 7-9, below) of 100 μM or less, more preferably 10 μM or less, and even more preferably 1 μM or less.

It is understood that while the pyrrole derivatives for use in the invention may exhibit the phenomenon of tautomerism, the formula drawings within this specification expressly depict only one of the possible tautomeric forms. It is therefore to be understood that within this specification the formulae are intended to represent any tautomeric form of the depicted compound, and the depicted compounds are not to be limited merely to a specific tautomeric form depicted by a formula drawing.

Some of the pyrrole derivatives for use in the invention may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in an optically pure form.

As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. Preferably, the pyrrole derivatives of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the Formulae presented above are intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of the Formulae I-VI, and those compounds specifically identified in Table 1, the invention includes pharmaceutically acceptable prodrugs, pharmaceutically active metabolites, and pharmaceutically acceptable salts of such compounds.

“A pharmaceutically acceptable prodrug” is a compound that may be converted under physiological conditions or by solvolysis to a specified compound of the Formulae I-VI, or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

Prodrugs and active metabolites of compound may be identified using routine techniques known in the art. See, e.g., Bertolini et al., J. Med. Chem., 40, 2011-2016 (1997); Shan et al., J. Pharm. Sci., 86 (7), 756-767; Bagshawe, Drug Dev. Res., 34, 220-230 (1995); Bodor, Advance in Drug Res., 13, 224-331 (1984); Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

“A pharmaceutically acceptable salt” is intended to mean a salt form of a compound of the Formulae I-VI, that retains the biological effectiveness of the free acids and bases of the specified compound and that is not biologically, physically, or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

The pyrrole derivatives of the present invention can have asymmetric centers and/or can exist in the form of cis or trans derivatives. The invention covers the racemates, mixtures of cis and trans compounds, and also covers optically active products with the cis derivatives and the trans derivatives taken independently. These pure products will be obtained by the methods known to those skilled in the art, in particular by chromatography, especially on chiral columns in the case of optical isomers, or, alternatively, by means of asymmetric synthetic protocols.

### Pharmaceutical Compositions

The present invention also provides pharmaceutical compositions comprising a therapeutic pyrrole derivative according to the present invention and a pharmaceutically acceptable excipient or carrier. Such pharmaceutical compositions are formulated so as to deliver a therapeutically or prophylactically effective amount of the therapeutic pyrrole derivative to a patient in need of such treatment.

When the composition having a compound of Formulae I-VI is administered, according to the treatment regimens of the invention, to an individual desiring or needing such treatment, it provides an improvement or lessening of a decline in symptoms associated with the disease or disorder exhibited by that patient. The pharmaceutical composition of the invention is formulated with one or more pharmaceutically acceptable salts, excipients, or carriers. The pharmaceutical composition can be delivered orally, preferably in a tablet or capsule dosage form, or by any other effective route. The pharmaceutical composition having a compound of Formulae I-VI can be used in methods for treating or preventing diseases or disorders characterized by the presence of axonal blocks, or the impairment of axonal transport or vesicle trafficking in neurons, or in the prophylaxis of such diseases or disorders in patients having increased risk of developing such diseases or disorders.

In a specific embodiment of this aspect of the invention, the dosage is provided as a pharmaceutical composition that is composed of an effective amount of a compound of Formulae I-VI, a pharmaceutically acceptable salt, a release agent, a carrier or excipient, and additional optional ingredients. In another specific embodiment of this aspect of the invention, the dosage is provided as a pharmaceutical composition that is a tablet composed of a compound of Formulae I-VI, microcrystalline cellulose, colloidal silicon dioxide, and magnesium stearate. In another specific embodiment of this aspect of the invention, the dosage is provided as a pharmaceutical composition comprising a compound of Formulae I-VI, microcrystalline cellulose, colloidal silicon dioxide, and magnesium stearate, all encapsulated in a pharmaceutically acceptable capsule, optionally including lactose monohydrate, hydroxylpropyl methyl cellulose, titanium dioxide, tracetin/glycerol triacetate, and iron oxide.

### Formulations

The pills, tablets, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the tablet-forming machine are selected by the skilled artisan.

### Therapeutic Methods

The present invention also provides therapeutic methods for use in treating patients in need of such treatments. These methods generally comprise administration of an effective amount of a pyrrole derivative of the present invention to a patient in need of such treatment, through the administration of a pharmaceutical composition of the present invention.

As a first step, the therapeutic methods of present invention require the identification of patients in need of such treatment. This first step can be achieved by way of any of the appropriate techniques known in the art, including assessment of symptoms, or the presence of specific biochemical defects or lesions.

A decline in sensory ability or fine motor control of an extremity, as observed in diseases and disorders such as ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; and DN, can be characterized by a variety of neurological tests. It is preferred that in patients treated with a pyrrole derivative of the present invention, an observed lessening in decline of sensory ability or fine motor control of an extremity is at least 25% as compared to individuals treated with placebo, more preferably at least 40%, and even more preferably at least 60%.

In certain embodiments, the present invention relates to a method of preventing diseases and disorders characterized by the presence of axonal blocks, or characterized by impaired axonal transport or impaired vesicle trafficking in neurons. According to this embodiment, a method for preventing such diseases and disorders is provided which comprises administering, to an individual in need of such treatment, a composition comprising a therapeutically effective amount of a compound according to Formulae I-VI. The method of this embodiment is useful for preventing or delaying the onset of the symptoms of diseases and disorders characterized by the presence of axonal blocks, or characterized by impaired axonal transport or impaired vesicle trafficking in neurons, the onset of such diseases and disorders, and/or the progression of such diseases and disorders. In these embodiments the patient in need of such treatment may be one who has yet to exhibit symptoms of such a disease or disorder, but is at risk of developing the disease or disorder. Alternatively, the patient to be treated may suffer from a mild form of such a disease or disorder, but has yet to be clinically diagnosed. Individuals at risk of developing such diseases and disorders can be identified by any acceptable method in the art. As noted above, such methods can include genotyping by any suitable method, analysis of family history of the disease, or through pedigree analysis. Methods of determining risk through genotyping include determining genotype by nucleic acid sequencing, selective hybridization, allele-specific amplification, and the like. Additionally, various biomarkers can be used to assess whether an individual is at risk of developing a disease or disorder that can be treated or prevented using the methods of the present invention.

### Patient Population

Any individual having, or suspected of having ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN can be treated using the compositions and methods of the present invention, as can individuals in which the promotion of axonal growth would be beneficial or therapeutic. Individuals who would particularly benefit from the compositions and methods of the invention include those individuals diagnosed as having mild to moderate ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN according to a medically-accepted diagnosis. Progression of the disease may be followed by medically accepted measures of impairment of sensory ability or fine motor control. Individuals diagnosed as having probable ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN can be identified by any recognized means of diagnosis in the art. In addition, methods that allow for evaluating different neuropathies can be used.

Diagnoses of ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN based on these tests are recorded as presumptive or probable, and may optionally be supported by one or more additional criteria. For example, a diagnosis of such diseases may be supported by evidence of a family history of the disease; non-specific changes in sensory ability or fine motor control of an extremity. Additionally, such associated symptoms can be used to make the diagnosis.

The invention encompasses the treatment of an individual having ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN, to the extent that individual has such a disease, whether or not one or more other neuropathies, neurodegenerative diseases, or conditions are previously, concurrently or subsequently diagnosed.

The compounds and methods of the present invention are useful for individuals who have received prior medication for their disease or disorder, as well as individuals who have received no prior medication, and are useful for individuals currently receiving medication for their disease or disorder other than a compound of the present invention, and for individuals not receiving medication for their disease or disorder other than a compound of the present invention.

Individuals of any age may be treated by the methods of the invention, with the pharmaceutical compositions of the invention; however, the invention encompasses specific embodiments for treating or preventing ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN in individuals between the ages of 35 and 100. In other various specific embodiments, individuals treated by the therapeutic or prophylactic methods of the invention may be from 35 to 40 years of age, 40 to 45 years of age, 45 to 50 years of age, 50 to 55 years of age, 55 to 60 years of age, 60 to 65 years of age, 65 to 70 years of age, 70 to 75 years of age, 75 to 80 years of age, or 80 years old and older.

Thus, in one embodiment, the invention provides a method of treating an individual known or suspected of having ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN comprising administering a therapeutically effective amount of a compound according to Formulae I-VI. In a specific embodiment, said individual is diagnosed as having mild to moderate ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN.

In yet another embodiment, the invention provides a method of slowing neurolopathological decline in an individual suspected of having mild ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN, comprising administering to the individual a therapeutically effective amount of a compound according to Formulae I-VI. In certain sub-embodiments, the invention provides a method of slowing neurolopathological decline in an individual through the promotion of axonal growth. Thus, according to one aspect of the invention, an individual suspected of having or diagnosed with ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN is treated twice daily with a composition having from 10 mg to about 1000 mg per dose of a compound of the present invention, either alone, or in combination with a therapeutically effective amount of another suitable therapeutic compound, for at least 4 weeks, at least 4 months, preferably at least 8 months, and more desirably at least 1 year.

The decline in sensory ability or fine motor control of an extremity in human patients can be characterized by any acceptable neurological test. It is preferred that the lessening in decline in sensory ability or fine motor control of an extremity is at least 25% as compared to individuals treated with placebo, at least 40%, or at least 60.

In other embodiments, the invention provides a method of treating an individual known or suspected of having ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN comprising administering an effective amount of a therapeutic compound of the present invention, wherein said individual is concurrently taking a second drug for the treatment of their disease or disorder. In a further embodiment, said individual has been diagnosed as having mild to moderate ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN.

In another embodiment, the individual to be treated with a pharmaceutical composition of the present invention is concurrently taking a non-pharmaceutical substance for the treatment of their disease or disorder along with a therapeutic compound of the present invention. In a specific embodiment, said non-pharmaceutical substance is an anti-oxidant. In a more specific example, said anti-oxidant is vitamin C or vitamin E. In an even more specific embodiment, said vitamin C is taken in a dose of 500-1000 mg per dose. In another even more specific embodiment, said vitamin E is taken in a dose of 400-800 IU per dose. In this regard, the invention encompasses the use of one or more such anti-oxidants as an adjunct to therapy for ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN, and not primarily as a nutritional supplement.

In another embodiment, the invention provides a method of treating an individual diagnosed as having ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN comprising administering an effective amount of a therapeutic compound of the present invention, wherein said individual has, prior to taking a therapeutic compound of the present invention, taken a second drug for the treatment of their disease or disorder.

Administration of a pharmaceutical composition of the present invention can be via any route, and the pharmaceutical compositions of the present invention can correspond to any compositions envisioned by one of skill in the art, appropriate to the route of delivery.

### Combination Therapy

The invention further provides a combination therapy strategy for treating or preventing ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN. According to this aspect of the invention, an individual in need of treatment is administered a therapeutic amount of a compound of the present invention according to Formulae I-VI, and a compound already approved for use in treating the disease or disorder that afflicts the individual in need of treatment.

The methods of combination therapy preferably provide a synergistic effect in reducing impaired axonal transport or impaired vesicle trafficking in neurons and are especially effective for preventing ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN. The treatment regimens used in the combination therapy can involve administration of pharmaceutical compositions comprising a combination of active ingredients, or the concomitant administration of separate compositions, each comprising at least one active ingredient. Furthermore, the administration of the active ingredients can be performed at different times and/or via different routes. For example, a composition comprising at least one active ingredient can be administered in the morning, and a composition comprising at least one different active ingredient can be administered in the evening. Another example would involve the administration of a composition having at least one active ingredient orally while the second composition having at least on other active ingredient is administered intravenously.

In addition to the advantages described above, while not wishing to be bound by theory, it is believed that therapeutic compounds of Formulae I-VI are capable of slowing the rate of death of neurons or slowing the atrophy of axonal processes caused by impaired axonal transport or impaired vesicle trafficking in neurons. Accordingly, it is also believed that the compounds of Formulae I-VI act in vivo to treat and/or prevent ALS; PD; CMT2; SMA; HSMN; LOSMA; poly Q diseases including Huntington disease, spinobulbar muscular atrophy, dentatorubral-pallidoluysian atrophy, Kennedy's disease (SBMA), spinocerebellar ataxia 1, spinocerebellar ataxia 2, spinocerebellar ataxia 3, spinocerebellar ataxia 6, spinocerebellar ataxia 7, and spinocerebellar ataxia 17; traumatic brain and spinal cord injury; HSP; MS; Guillain-Barré syndrome; PLS; taupathies including supranuclear palsy, corticobasal degeneration, Pick's disease, argyrophilic grain disease, and frontotemporal dementia and parkinsonism linked to chromosome 17 (FTDP-17); DLB; NPC; optic neuropathies including LHON and CEON; or DN by alleviating the impaired axonal transport or alleviating the impaired vesicle trafficking in neurons that is present, or would be present, in the absence of such treatment.

### EXAMPLES

### Example 1

### Synthesis of Reaction Material

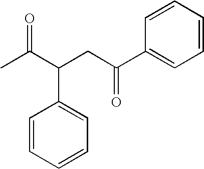

1,3-Diphenyl-pentane-1,4-dione: 1-Trimethylsilanyl-ethanone (1.15 mL; 8.02 mmol) followed by DBU (0.18 mL; 1.2 mmol) were added to a suspension of 3-ethyl-5-(2-hydroxyethyl)-4-methylthiazolium bromide (304 mg; 1.21 mmol) in dry THF (5 mL). The mixture was heated at 70° C. for 4 min, cooled near rt, then chalcone (833 mg; 4.00 mmol) and 2-propanol (1.22 mL; 15.9 mmol) were added. The reaction was degassed and reacted under nitrogen at 70° C. After 24 h, the reaction was concentrated on a rotary evaporator. Ethyl acetate (25 mL) was added and this washed with H2O (3×3 mL) and satd NaCl (2×3 mL). The organic portion was dried (MgSO4) and filtered through silica with an EtOAc wash. Crude product was adsorbed onto silica (2 g) then purified by MPLC (40 g of silica using a 0→20% EtOAc in hexanes gradient). Pure product was obtained as a clear, colorless viscous liquid (980 mg; 97%). ¹H NMR (CDCl3) δ 7.98-7.94 (m, 2H), 7.56 (m, 1H), 7.47-7.42 (m, 2H), 7.39-7.27 (m, 5H), 4.44 (dd, J=3.6, 10.0 Hz, 1H), 4.02 (dd, J=10.0, 18.0 Hz, 1H), 3.14 (dd, J=3.6, 18.0 Hz, 1H); GC-MS 252 ([M]+).

### Example 2

### Synthesis of 4-[4-(2-Methyl-3,5-diphenyl-pyrrol-1-yl)-phenyl]-butyric acid

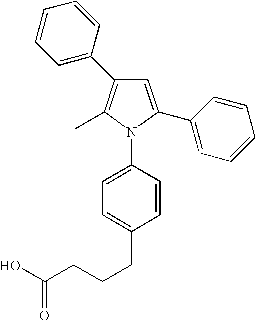

4-[4-(2-Methyl-3,5-diphenyl-pyrrol-1-yl)-phenyl]-butyric acid: A soln of 1,3-diphenyl-pentane-1,4-dione (104 mg; 0.412 mmol) and 4-(4-amino-phenyl)butyric acid (89 mg; 0.497 mmol) in acetic acid (2 mL) was heated at 120° C. After 6 h, the reaction was concentrated on a rotary evaporator. Ethyl acetate (5 mL) was added and this washed with H2O (1×2 mL) and satd NaCl (1×3 mL). The organic portion was dried (MgSO4) and filtered through silica with an EtOAc wash. Crude product was adsorbed onto silica (0.3 g) then purified by MPLC (12 g of silica using a 0→50% EtOAc in hexanes gradient). Pure product was obtained as a white solid (115 mg; 71%). ¹H NMR (CDCl3) δ 7.54-7.49 (m, 2H), 7.44-7.38 (m, 2H), 7.26-7.07 (m, 10H), 6.56 (s, 1H), 2.72 (t, J=7.6 Hz, 2H), 2.40 (t, J=7.4 Hz, 2H), 2.25 (s, 3H), 2.00 (m, 2H); LC-MS (ESI−) 394 ([M-H]−).

### Example 3

### Synthesis of Selected Example Compounds

Using the general reaction schemes presented in Example 2, above, compounds of the present invention can be synthesized from the starting materials identified in Table 2, below.

TABLE 2
Starting Materials and Example Compounds Synthesized

| Cmpd. | Compound | Ketone/Aldehyde | | | |
|---|---|---|---|---|---|
| No. | Structure | starting material | Aniline | 1H NMR, δ | MS |
| 1 | 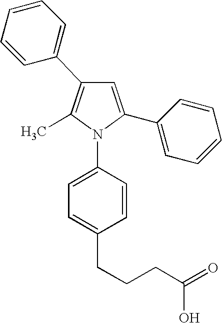 | 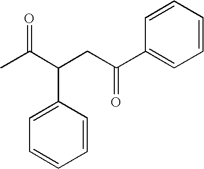 | 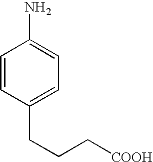 | CDCl3: 7.54- 7.49 (m, 2H), 7.44-7.38 (m, 2H), 7.26-7.07 (m, 10H), 6.56 (s, 1H), 2.72 (t, J = 7.6 Hz, 2H), 2.40 (t, J = 7.4 Hz, 2H), 2.25 (s, 3H), 2.00 (m, 2H) | 394 ([M − H]−) |
| 2 | 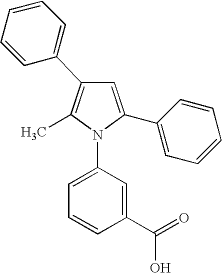 | 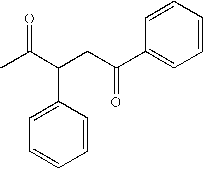 | 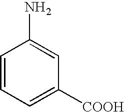 | DMSO-d6: 7.97 (m, 1H), 7.70 (m, 1H), 7.64- 7.55 (m, 2H), 7.53-7.48 (m, 2H), 7.45-7.40 (m, 2H), 7.27- 7.16 (m, 3H), 7.14-7.06 (m, 3H), 6.63 (s, 1H) | 352 ([M − H]−) |
| 3 | 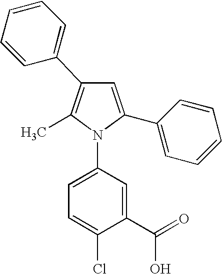 | 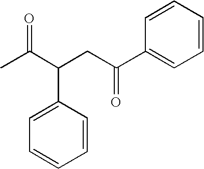 | 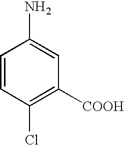 | CDCl3: 7.96 (d, J = 2.4 HZ, 1H), 7.52-7.46 (m, 3H), 7.45- 7.40 (m, 2H), 7.30-7.09 (m, 7H), 6.56 (s, 1H), 2.27 (s, 3H) | 388 ([M + H]+) |
| 4 | 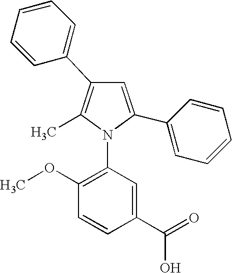 | 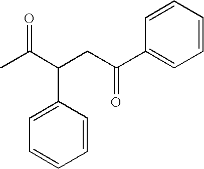 | 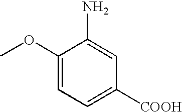 | CDCl3: 8.12 (dd, J = 2.2, 8.6 Hz, 1H), 7.91 (d, J = 2.4 Hz, 1H), 7.55-7.50 (m, 2H), 7.44- 7.38 (m, 2H), 7.27-7.21 (m, 1H), 7.18-7.07 (m, 5H), 7.02 (d, J = 8.8 Hz, 1H), 6.57 (s, 1H), 3.77 (s, 3H), 2.19 (s, 3H) | 384 ([M + H]+) |
| 5 | 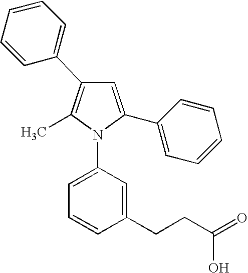 | 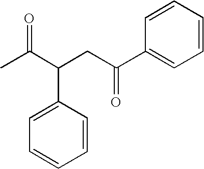 | 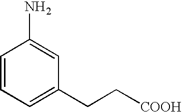 | CDCl3: 7.53- 7.49 (m, 2H), 7.44-7.38 (m, 2H), 7.32 (m, 1H), 7.28-7.06 (m, 8H), 7.03 (m, 1H), 6.56 (s, 1H), 2.92 (t, J = 7.4 Hz, 2H), 2.56 (t, J = 7.6 Hz, 2H), 2.25 (s, 3H) | 380 ([M − H]−) |
| 6 | 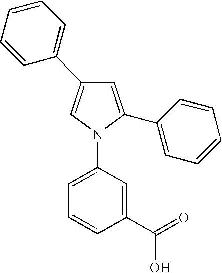 | 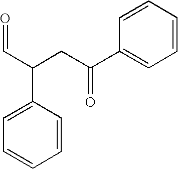 | 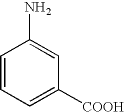 | CDCl3: 8.07 (m, 1H), 8.02 (m, 1H), 7.61 (m, 2H), 7.43- 7.16 (m, 11H), 6.77 (d, J = 1.6 Hz, 1H) | 340 ([M + H]+) |
| 7 | 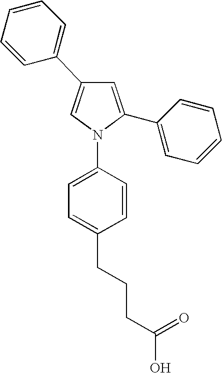 | 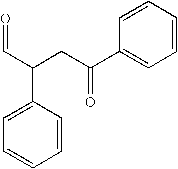 | 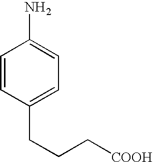 | CDCl3: 7.62- 7.57 (m, 2H), 7.40-7.34 (m, 2H), 7.25-7.11 (m, 11H), 6.74 (d, J = 2.0 Hz, 1H), 2.69 (t, J = 7.6 Hz, 2H), 2.39 (t, J = 7.2 Hz, 2H), 1.98 (m, 2H) | 382.17974 (TOF; [M + H]+) |
| 8 | 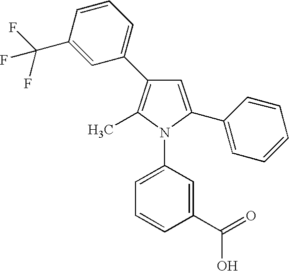 | 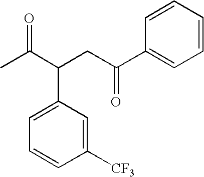 | 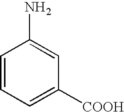 | CDCl3: 8.11 (m, 1H), 8.04 (m, 1H), 7.75 (m, 1H), 7.67 (m, 1H), 7.56- 7.47 (m, 3H), 7.41 (m, 1H), 7.22-7.08 (m, 5H), 6.58 (s, 1H), 2.26 (s, 3H) | 420 ([M − H]−) |
| 9 | 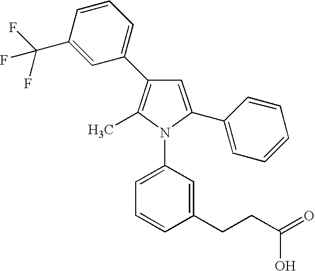 | 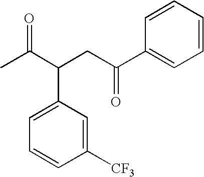 | 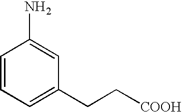 | CDCl3: 7.75 (m, 1H), 7.67 (m, 1H), 7.54- 7.46 (m, 2H), 7.33 (m, 1H), 7.22-7.02 (m, 8H), 6.56 (s, 1H), 2.92 (t, J = 7.4 Hz, 2H), 2.56 (t, J = 7.4 Hz, 2H), 2.24 (s, 3H) | 450.16704 (TOF; [M + H]+) |
| 10 | 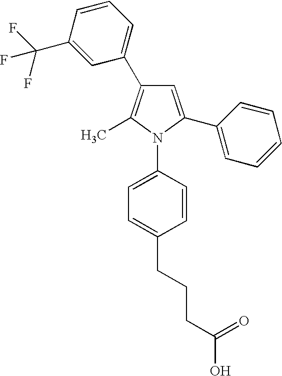 | 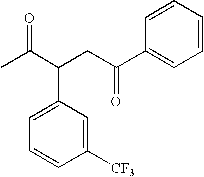 | 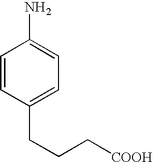 | CDCl3: 7.75 (m, 1H), 7.67 (m, 1H), 7.54- 7.46 (m, 2H), 7.24-7.08 (m, 9H), 6.56 (s, 1H), 2.72 (t, J = 7.6 Hz, 2H), 2.40 (t, J = 7.4 Hz, 2H), 2.24 (s, 3H), 2.00 (m, 2H) | 462 ([M − H]−) |
| 11 | 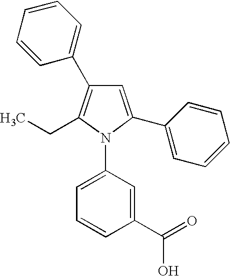 | 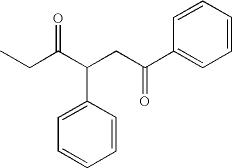 | 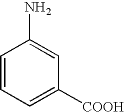 | CDCl3: 8.14- 8.08 (m, 2H), 7.53-7.38 (m, 6H), 7.30-7.24 (m, 1H), 7.19- 7.06 (m, 5H), 6.55 (s, 1H), 2.71 (q, J = 7.2 Hz, 2H), 0.92 (t, J = 7.6 Hz, 3H) | 368.16533 (TOF; [M + H]+) |
| 12 | 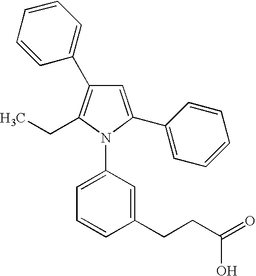 | 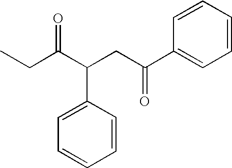 | 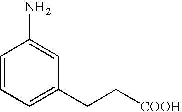 | CDCl3: 7.53- 7.48 (m, 2H), 7.43-7.37 (m, 2H), 7.32 (m, 1H), 7.28-7.18 (m, 2H), 7.16- 7.04 (m, 7H), 6.53 (s, 1H), 2.92 (t, J = 7.6 Hz, 2H), 2.71 (q, J = 7.5 Hz, 2H), 2.57 (t, J = 7.6 Hz, 2H), 0.90 (t, J = 7.6 Hz, 3H) | 396.19522 (TOF; [M + H]+) |
| 13 | 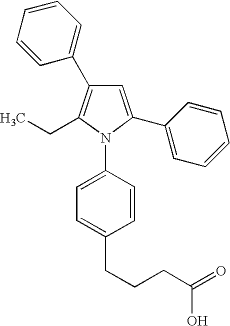 | 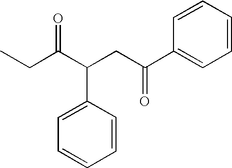 | 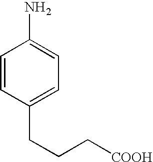 | CDCl3: 7.53- 7.49 (m, 2H), 7.43-7.37 (m, 2H), 7.28-7.06 (m, 10H), 6.54 (s, 1H), 2.76- 2.66 (m, 4H), 2.39 (t, J = 7.4 Hz, 2H), 2.00 (m, 2H), 0.92 (t, J = 7.4 Hz, 3H) | 432.19283 (TOF; [M + Na]+) |
| 14 | 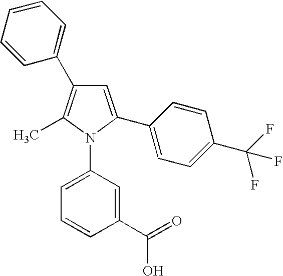 | 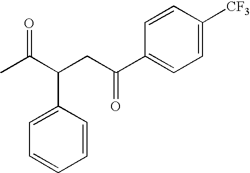 | 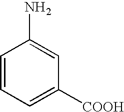 | CDCl3: 8.14 (m, 1H), 8.05 (m, 1H), 7.56- 7.38 (m, 8H), 7.31-7.25 (m, 1H), 7.20-7.15 (m, 2H), 6.65 (s, 1H), 2.26 (s, 3H) | 422.13646 (TOF; [M + H]+) |
| 15 | 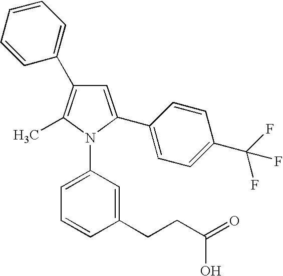 | 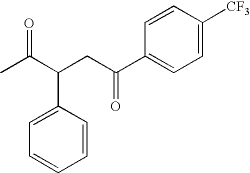 | 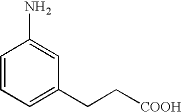 | CDCl3: 7.52- 7.47 (m, 2H), 7.44-7.31 (m, 5H), 7.29-7.20 (m, 2H), 7.18- 7.14 (m, 2H), 7.09-7.07 (m, 2H), 6.64 (s, 1H), 2.95 (t, J = 7.4 Hz, 2H), 2.60 (t, J = 7.4 Hz, 2H), 2.24 (s, 3H) | 450.16678 (TOF; [M + H]+) |
| 16 | 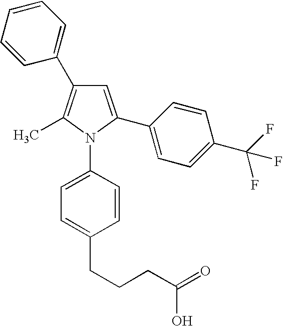 | 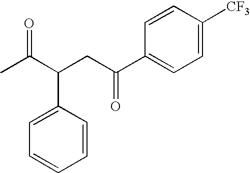 | 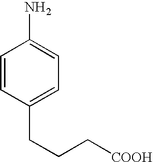 | CDCl3: 7.52- 7.47 (m, 2H), 7.45-7.36 (m, 4H), 7.29-7.21 (m, 3H), 7.19- 7.13 (m, 4H), 6.64 (s, 1H), 2.74 (t, J = 8.0 Hz, 2H), 2.41 (t, J = 7.6 Hz, 2H), 2.24 (s, 3H), 2.02 (m, 2H) | 486.16389 (TOF; [M + Na]+) |
| 17 | 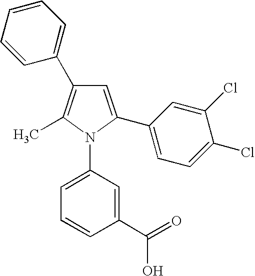 | 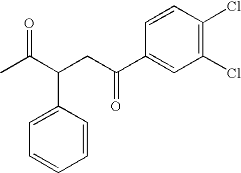 | 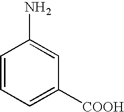 | CDCl3: 8.14 (m, 1H), 8.01 (m, 1H), 7.55 (m, 1H), 7.50- 7.40 (m, 5H), 7.31-7.25 (m, 2H), 7.18 (d, J = 8.4 Hz, 1H), 6.79 (dd, J = 2.4, 8.4 Hz, 1H), 6.59 (s, 1H), 2.24 (s, 3H) | 422.07034 (TOF; [M + H]+) |
| 18 | 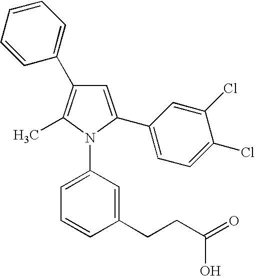 | 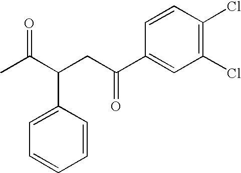 | 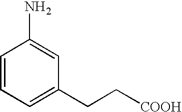 | CDCl3: 7.50- 7.46 (m, 2H), 7.44-7.32 (m, 3H), 7.28-7.22 (m, 2H), 7.20- 7.15 (m, 2H), 7.10-7.04 (m, 2H), 6.81 (dd, J = 2.0, 8.4 Hz, 1H), 6.58 (s, 1H), 2.97 (t, J = 7.6 Hz, 2H), 2.64 (t, J = 7.4 Hz, 2H), 2.22 (s, 3H) | 450.10227 (TOF; [M + H]+) |
| 19 | 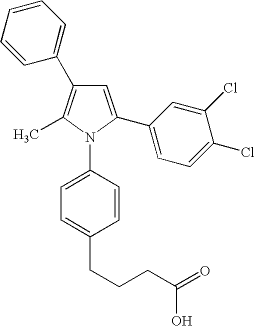 | 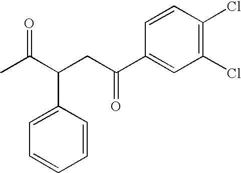 | 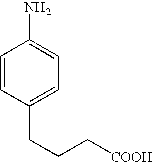 | CDCl3: 7.52- 7.46 (m, 2H), 7.44-7.38 (m, 2H), 7.28-7.23 (m, 3H), 7.20- 7.11 (m, 4H), 6.86 (dd, J = 2.0, 8.4 Hz, 1H), 6.58 (s, 1H), 2.75 (t, J = 7.6 Hz, 2H), 2.41 (t, J = 7.6 Hz, 2H), 2.24 (s, 3H), 2.02 (m, 2H) | 462 ([M − H]−) |
| 20 | 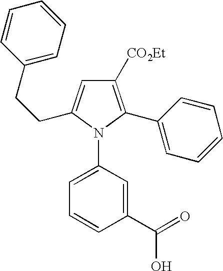 | 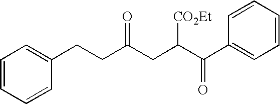 | 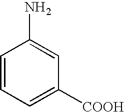 | CDCl3: 7.99 (m, 1H), 7.79 (m, 1H), 7.35 (m, 1H), 7.26- 7.20 (m, 2H), 7.20-7.11 (m, 7H), 7.04 (m, 2H), 6.69 (m, 1H), 4.17 (q, J = 7.2 Hz, 2H), 2.86 (t, J = 7.8 Hz, 2H), 2.69 (m, 2H), 1.17 (t, J = 7.2 Hz, 3H) | 440 ([M + H]+) |
| 21 | 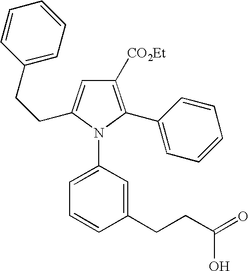 | 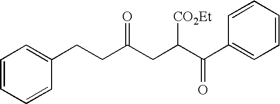 | 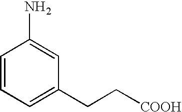 | CDCl3: 7.26- 7.10 (m, 9H), 7.08 (m, 1H), 7.03 (m, 2H), 6.88 (m, 1H), 6.76 (m, 1H), 6.66 (m, 1H), 4.16 (q, J = 7.1 Hz, 2H), 2.86- 2.76 (m, 4H), 2.69 (m, 2H), 2.43 (t, J = 7.6 Hz, 2H), 1.17 (t, J = 7.2 Hz, 3H) | 468 ([M + H]+) |
| 22 | 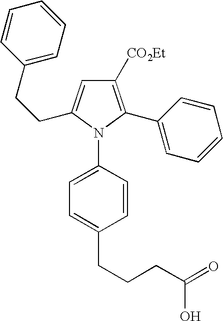 | 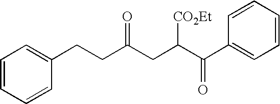 | 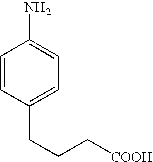 | CDCl3: 7.26- 7.20 (m, 2H), 7.19-7.12 (m, 6H), 7.08-7.01 (m, 4H), 6.90 (m, 2H), 6.65 (m, 1H), 2.83 (m, 2H), 2.68 (m, 2H), 2.62 (m, 2H), 2.31 (t, J = 7.2 Hz, 2H), 1.91 (m, 2H), 1.17 (t, J = 7.2 Hz, 3H) | 482 ([M + H]+) |
| 23 | 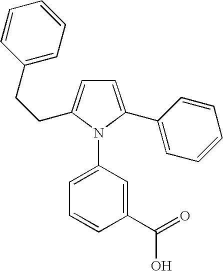 | 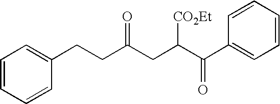 | 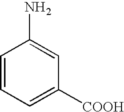 | CDCl3: 8.03 (m, 1H), 7.93 (m, 1H), 7.40 (m, 1H), 7.26- 7.01 (m, 11H), 6.39 (d, J = 3.2 Hz, 1H), 6.19 (d, J = 3.6 Hz, 1H), 2.83-2.72 (m, 4H) | 368.16386 (TOF; [M + H]+) |
| 24 | 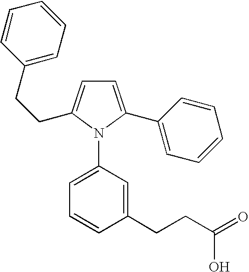 | 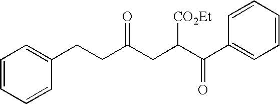 | 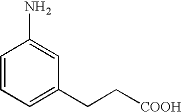 | CDCl3: 7.31- 7.00 (m, 13H), 6.92 (m, 1H), 6.39 (d, J = 3.6 Hz, 1H), 6.16 (d, J = 3.2 Hz, 1H), 2.86 (t, J = 7.6 Hz, 2H), 2.84-2.72 (m, 4H), 2.50 (t, J = 7.6 Hz, 2H) | 396.19502 (TOF; [M + H]+) |
| 25 | 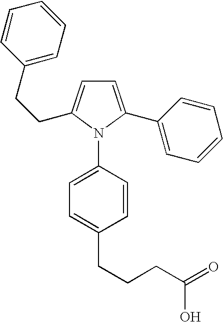 | 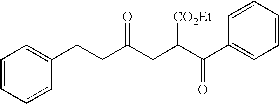 | 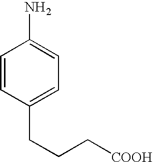 | CDCl3: 7.26- 7.20 (m, 14H), 6.39 (d, J = 3.6 Hz, 1H), 6.17 (m, 1H), 2.84- 2.73 (m, 4H), 2.70 (t, J = 7.6 Hz, 2H), 2.38 (t, J = 7.4 Hz, 2H), 1.98 (m, 2H) | 410.21222 (TOF; [M + H]+) |
| 26 | 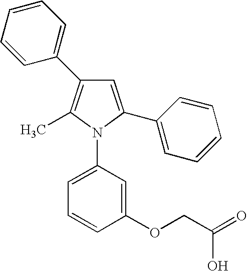 | 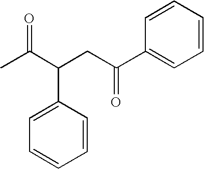 | 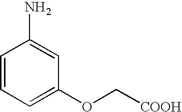 | CDCl3: 7.52- 7.48 (m, 2H), 7.41 (m, 2H), 7.33 (m, 1H), 7.28-7.08 (m, 6H), 6.96-6.91 (m, 2H), 6.74 (m, 1H), 6.55 (s, 1H), 4.58 (s, 2H), 2.26 (s, 3H) | 384.16033 (TOF; [M + H]+) |
| 27 | 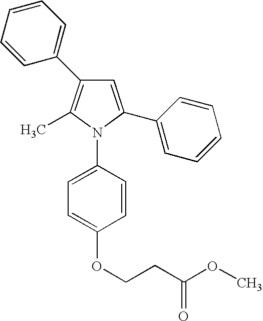 | 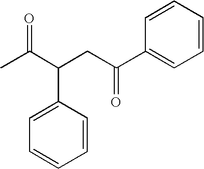 | 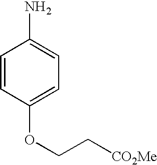 | CDCl3: 7.53- 7.49 (m, 2H), 7.41 (m, 2H), 7.24 (m, 1H), 7.20-7.08 (m, 7H), 6.90 (m, 2H), 6.55 (s, 1H), 4.27 (t, J = 6.4 Hz, 2H), 3.75 (s, 3H), 2.83 (t, J = 6.0 Hz, 2H), 2.23 (s, 3H) | <cmpd did not ionize for electrospray MS> |
| 28 | 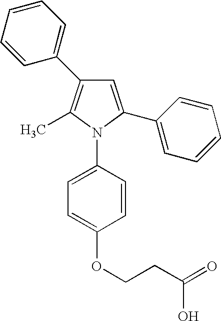 | 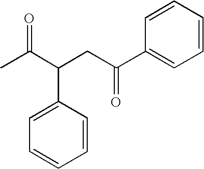 | 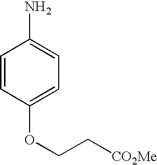 | CDCl3: 7.53- 7.48 (m, 2H), 7.41 (m, 2H), 7.24 (m, 1H), 7.20-7.08 (m, 7H), 6.91 (m, 2H), 6.55 (s, 1H), 4.27 (t, J = 6.4 Hz, 2H), 2.89 (t, J = 6.2 Hz, 2H), 2.23 (s, 3H) | 396 ([M − H]−) |

### Example 4

### Pressed Tablet Formulations

| Ingredient | Amount | Preferred Ranges |
|---|---|---|
| Compound of Formulae I-VI | 400 mg | +50% to −50% |
| Microcrystalline Cellulose | 392 mg | +50% to −50% |
| Colloidal Silicon Dioxide | 4 mg | +50% to −50% |
| Magnesium Stearate | 4 mg | +50% to −50% |

The tablets are prepared using art known procedures.

### Example 5

### Coated Tablet Formulations

| Ingredient | Amount | Preferred Ranges |
|---|---|---|
| Compound of Formulae I-VI | 400 mg | +50% to −50% |
| Microcrystalline Cellulose | 392 mg | +50% to −50% |
| Colloidal Silicon Dioxide | 4 mg | +50% to −50% |
| Magnesium Stearate | 4 mg | +50% to −50% |
| Coated with: | | |
| Lactose monohydrate | | |
| Hydroxyl propyl methyl cellulose | | |
| Titanium dioxide | | |
| Tracetin/glycerol triacetate | | |
| Iron oxide | | |

The coated tablets are produced using art known procedures.

### Example 6

### Capsule Formulations

| Ingredient | Amount | Preferred Ranges |
|---|---|---|
| Compound of Formulae I-VI | 400 mg | +50% to −50% |
| Microcrystalline Cellulose | 392 mg | +50% to −50% |
| Colloidal Silicon Dioxide | 4 mg | +50% to −50% |
| Magnesium Stearate | 4 mg | +50% to −50% |
| Encapsulated in gelatin | | |

The capsules are produced using art known procedures.

### Example 7

### Animal Model for Axonal Vesicular Transport Blockage and Motor Dysfunction

A stock of fruit flies (Drosophila melanogaster) was generated that is heterozygous for both KHC and KLC, which encode proteins that associate to form functional kinesin-1, also called conventional kinesin. As a result of the approximately 50% reduction in the level of kinesin-1, these khc/+; klc/+ larvae exhibit a motor defect termed “tail-flipping”. Specifically, the mutant larvae exhibit loss of motor activity in the ventral posterior segments that causes an imbalance in body wall contractions; as a result, the larvae rhythmically flip their tails upward during locomotion. In preliminary studies the penetrance of the tail-flipping phenotype was found to be less than 100%; that is, not all khc/+; klc/+ larvae showed the phenotype. A number of factors that contribute to this incomplete penetrance were identified, including the following:
1. The flipper phenotype of a given animal appears to be suppressed by the number of larvae that precede the animal in development. That is, if a larva is among the first to develop in a vial of eggs, it is more likely to show the flipper phenotype than if it is one of the last to emerge.
2. The flipper phenotype appears to be less robust on hard media than it is on soft media.
3. The phenotype is diminished by physically disturbing the larvae.
4. The clearest expression of the flipper phenotype is restricted to that phase of the 3^{rd} instar stage of development that follows the appearance of spiracles.

Attempts to accommodate these observations were mad in order to optimize penetrance of the phenotype. Specifically:
1. Virgin females and males were confined to a single vial for only 2 days; the flies were then transferred to fresh vials for an additional 2 days; and this process was repeated to minimize the number of larvae that would emerge in each vial.
2. Efforts were taken to minimize handling of the larvae.
3. Attempts were made to score the phenotype late in the 3rd instar stage of development.

After these optimization steps were taken the penetrance of the phenotype appeared to be consistent with literature values (See Martin et al., Mol. Cell. Biol. 10:3717-3728 (1999)).

Importantly, the flipper phenotype of khc/+; klc/+ Drosophila larvae is considered to be a model of some human motor neuropathies (e.g., diseases associated with a defect in vesicular transport), including certain forms of ALS (Hurd and Saxton, Genetics 144:1075-1085 (1996)). Indeed, the relevance of the Drosophila model to ALS is supported by a recent report using the SOD1^{G93A} mouse model of ALS (Kieran et al., J. Cell Biol. 169:561-567 (2005)). This report showed amelioration of disease when the ALS-prone mice were made mutant for the dynein heavy chain. This result, which is paradoxical on several grounds, was anticipated by dynein mutations in Drosophila models of ALS (Gunawardena and Goldstein, Neuron 32:389-401 (2001)).

### Example 8

### Treatment of Motor Dysfunction in an Animal Model

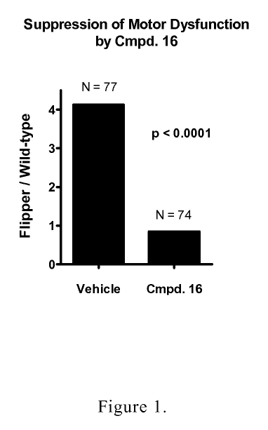

In a non-blinded experiment, compound 16 (Tables 1 and 2) was tested for its ability to suppress the flipper phenotype of khc/+; klc/+ Drosophila larvae (using the animal model described in Example 7), relative to vehicle (DMSO) alone. Specifically khc/+; klc/+ Drosophila larvae were grown in the presence or absence of 0.5 mM compound 16 and scored for motor dysfunction. A total of 151 larvae were scored, 77 in the vehicle-only group and 74 in the vehicle plus compound group. Results (FIG. 1) are expressed in terms of the number of flies exhibiting some degree of motor dysfunction (Flipper) relative to the number with no observable dysfunction (Non-Flipper or Wild-type) for the vehicle-only group (Vehicle) and the vehicle plus compound 16 group (Cmpd. 16; treatment group). The ratio of Flipper to Non-Flipper phenotype observed in the vehicle-only group corresponds to an 81% penetrance of the flipper phenotype in this experiment. Fischer's test for difference between the two conditions (FIG. 1) indicates a p value less than 10⁻⁴, indicating significant suppression of the Flipper phenotype by the compound. Note that equivalent conclusions were obtained if, instead of scoring the number of larvae with and without motor dysfunction, the severity of the aberrant movement was analyzed.

This experiment was repeated using a blinded format and a total of 221 larvae were scored. Of the larvae in this study 104 larvae were in the vehicle-only group and 117 larvae were in the treatment group. After the results were obtained and the study was unblended, a Yates' chi-square test indicated that treated group had a highly significant reduction in flipper phenotype (data not shown), with a p value of <1×10⁻⁶ (p<0.000001). The results of this blinded study, in which penetrance of the flipper phenotype in the vehicle-only control was 78%, clearly confirmed that compound 16 was able to significantly suppress motor dysfunction in the khc/+; klc/+ Drosophila larvae. The results of this second study are quite similar to those of the first study: after accounting for the differences in penetrance between the studies, compound 16 effectively “cured” 42% of the larvae of their motor dysfunction in the first experiment, and 43% of the larvae in the second experiment.

It is noteworthy that the penetrance of the flipper phenotype in these experiments (81% and 78%) was greater than that observed by others (50-70%) in previously published studies of khc/+; klc/+ Drosophila larvae (Martin et al. Mol. Biol. Cell 10:3717-3728 (1999)). The particularly high penetrance observed in the experiment reported here may stem from improvements in growing and handling the khc/+; klc/+ Drosophila larvae that served to maximize penetrance of the phenotype, and reproducibility of experimental results.

In conclusion, both of the studies discussed above support the conclusion that certain pyrrole derivatives (for example, compound 16) can rescue a motor dysfunction in Drosophila that results from impaired axonal transport.

In view of the predictive power of Drosophila models for interventions that ameliorate ALS, the results obtained in these studies suggest that the pyrrole derivatives of the present invention can be used to treat ALS, and other neuropathies characterized by the presence of axonal blockages, impaired axonal transport, or impaired vesicle trafficking in neurons. Thus it is believed that the compounds of the invention can be used to alleviate axonal blockages, alleviate impaired axonal transport, or alleviate impaired vesicle trafficking in neurons, and thereby treat diseases and disorders such as ALS, that are associated with such defects.

### Example 9

### Alleviation of Axonal Blockages (Jams) in an Animal Model

The flipper phenotype of khc/+; klc/+ Drosophila larvae is tightly correlated with the presence of axonal blockages (jams) comprising accumulated microtubule-associated and motor proteins, organelles, and vesicles (Hurd and Saxton, Genetics 144:1075-1085 (1996); Horiuchi et al., Curr. Biol. 15:2137-2141 (2005)). Therefore, it is reasonable to predict that khc/+; klc/+ Drosophila larvae exposed to the pyrrole derivatives of the present invention will have fewer, or less severe, axonal jams than larvae exposed to vehicle only. This prediction can be tested by growing khc/+; klc/+ Drosophila larvae on medium containing the pyrrole derivatives of the present invention, and conducting specific histological examinations for axonal jams. Specifically, larvae can be raised under the conditions outlined in Example 8, fixed in formaldehyde, and used to prepare larval pelts, using variations on established procedures. The larval pelts can then be treated with antibodies against synaptotagmin, which serves to label the secretory vesicles in the formaldehyde-fixed neurons, and subsequently labeled with a fluorescent tag, again using variations on established procedures. The treated larval pelts can then be subjected to immunohistological examination, and the labeled axonal jams can be imaged and quantitated by confocal fluorescent microscopy. Both the numbers and sizes of the axonal jams can be quantitated and comparisons can be made, and statistically evaluated, between test larvae exposed to the pyrrole derivatives of the present invention, and control larvae exposed only to liquid vehicle.

### Example 10

### Treatment of ALS with a Compound of Formulae I-VI

A therapeutic compound of Formulae I-VI can be used to treat ALS by administering tablets containing 50 mg of the compound, and/or oral gel capsules containing 50 mg of the compound. The typical dosage may be 50, 100, 300 or 600 mg of active ingredients daily. A typical dosage regimen may have 100 mg of the compound taken daily (50 mg twice daily). Another typical dosage may have 50 mg of the compound taken once daily. These dosages can also be divided or modified, and taken with or without food. For example, a 200 mg dose can be divided into two 100 mg tablets or capsules.

Depending on the stage of the disease, the therapeutic compound of Formulae I-VI can also be administered once daily in liquid, capsule, or tablet dosage forms where the dose has various amounts of compound (i.e., 300 mg, 250 mg, 200 mg, 175 mg, 150 mg, 125 mg, 100 mg, 75 mg, 50 mg, 40 mg, 30 mg, 25 mg, 15 mg, 10 mg and 1 mg). Again, the dosages can also be divided or modified, and taken with or without food. The doses can be taken during treatment with other medications for treating ALS or symptoms thereof.

Patients having mild-to-moderate ALS undergoing the treatment regimen of this example with a therapeutic compound of Formulae I-VI in doses of about 1 mg to 400 mg can experience a lessening in decline of motor function and/or biochemical disease marker progression.

### Example 11

### Prevention of ALS

Prior to the onset of symptoms of ALS or just at the very beginning stages of the disease, patients desiring prophylaxis against ALS can be treated with a prophylactically effective amount of a therapeutic compound of Formulae I-VI. Those needing prophylaxis can be assessed by monitoring assayable disease markers, detection of genes conferring a predisposition to the disease, other risks factors such as age, diet, other disease conditions associated with ALS. The patient can also be treated with a combination of NMDA, and a therapeutic compound of Formulae I-VI to delay or prevent the onset of ALS or symptoms thereof.

The patient desiring prophylaxis against AD or prophylaxis of a worsening of the symptoms of ALS can be treated with a therapeutic compound of Formulae I-VI in an amount sufficient to delay the onset or progression of symptoms of ALS. For example, a patient can be treated with 100 mg of a compound of Formulae I-VI once daily. Another preventive regimen involves administering to the patient 50 mg of compound of Formulae I-VI once daily. These amounts of these active ingredients can be modified to lessen side-effects and/or produce the most therapeutic benefit. For example, 25 mg of a therapeutic compound of Formulae I-VI twice daily can be administered to reduce side-effects associated with the use of higher levels of the active ingredient. The preventive treatment can also be, e.g., treatment on alternating days with compound of Formulae I-VI or alternating weeks. Other preventive treatment regimens include, but are not limited to, treatment with compound of Formulae I-VI for 3 weeks out of every 4 weeks, or for several months followed by no treatment for a month and then treatment for several months in an alternating on/off schedule to reduce side effects or toxicity problems.

Patients desiring or in need of prophylaxis against ALS undergoing the preventive regimen of this example with a therapeutic compound of Formulae I-VI doses of about 1 mg to 400 mg can decelerate or delay the onset of ALS or prevent the occurrence of ALS. It can be advantageous to utilize a low dosage prevention regimen that involves administration of pharmaceutical doses of 50 mg compound of Formulae I-VI once daily.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1. (canceled)

2. A method of treating a neurodegenerative disease characterized by the occurrence of axonal blockages in a human patient comprising identifying a patient in need of such treatment and administering to said patient a therapeutically effective amount of a compound having a structure according to Formula I: 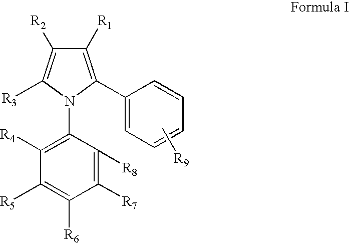
or pharmaceutically acceptable salts thereof, wherein:
R₁ is a hydrogen atom, halogen, hydroxy, alkyl, substituted alkyl, alkoxy, substituted alkoxy, or —CO₂R₁₀, and R₁₀ is alkyl or substituted alkyl;
R₂ is a hydrogen atom, halogen, hydroxy, alkyl, substituted alkyl, alkoxy, substituted alkoxy, or phenyl, optionally substituted with 0-5 phenyl substituents;
R₃ is a hydrogen atom, halogen, hydroxy, alkyl, substituted alkyl, alkoxy, or substituted alkoxy when R₂ is phenyl, or, when R₂ is not phenyl R₃ is —CH₂CH₂-phenyl optionally substituted with 0-5 phenyl substituents;
R₄ is a hydrogen atom, halogen, hydroxy, alkyl, substituted alkyl, alkoxy, or substituted alkoxy;
R₅ is a hydrogen atom, halogen, hydroxy, alkyl, substituted alkyl, alkoxy, or substituted alkoxy;
either of R₆ or R₇ is —(CH₂)ₙCO₂H or —O(CH₂)ₙCO₂H, wherein n is an integer from 0 to 4, or —(CH₂)ₘ—O—(CH₂)ₚCO₂H, wherein m is an integer from 1 to 2 and p is an integer from 1 to 2, while the other of R₆ or R₇ is a hydrogen atom, halogen, hydroxy, alkyl, substituted alkyl, alkoxy, or substituted alkoxy;
R₈ is a hydrogen atom, halogen, hydroxy, alkyl, substituted alkyl, alkoxy, or substituted alkoxy; and
R₉ is 0-5 phenyl substituents selected from halogen, hydroxy or haloalkyl.

3. The method of claim 2, wherein said neurodegenerative disease is amyotrophic lateral sclerosis, primary lateral sclerosis, progressive muscular atrophy, pseudobulbular palsy, progressive bulbular palsy, spinal muscular atrophy, spinobulbar muscular atrophy, multiple sclerosis, Parkinson's disease, dementia with Lewy bodies, Charcot-Marie-Tooth disease (type 2A), hereditary spastic paraplegia, Guillain-Barré syndrome; Huntington disease, dentatorubral-pallidoluysian atrophy, spinocerebellar ataxia 1, spinocerebellar ataxia 2, spinocerebellar ataxia 3, spinocerebellar ataxia 6, spinocerebellar ataxia 7, spinocerebellar ataxia 17, supranuclear palsy, corticobasal degeneration, Pick's disease, argyrophilic grain disease, frontotemporal dementia, parkinsonism linked to chromosome 17, or Niemann-Pick type C disease.

4. A method of treating a neuropathy associated with a defect in axonal transport in a human patient comprising identifying a patient in need of such treatment and administering to said patient a therapeutically effective amount of a compound having a structure according to Formula I: 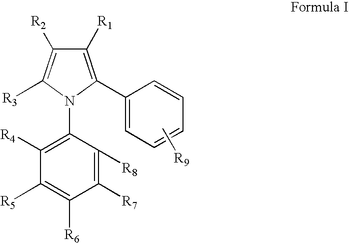
or pharmaceutically acceptable salts thereof, wherein:
R₁ is a hydrogen atom, halogen, hydroxy, alkyl, substituted alkyl, alkoxy, substituted alkoxy, or —CO₂R₁₀, and R₁₀ is alkyl or substituted alkyl;
R₂ is a hydrogen atom, halogen, hydroxy, alkyl, substituted alkyl, alkoxy, substituted alkoxy, or phenyl, optionally substituted with 0-5 phenyl substituents;
R₃ is a hydrogen atom, halogen, hydroxy, alkyl, substituted alkyl, alkoxy, or substituted alkoxy when R₂ is phenyl, or, when R₂ is not phenyl R₃ is —CH₂CH₂-phenyl optionally substituted with 0-5 phenyl substituents;
R₄ is a hydrogen atom, halogen, hydroxy, alkyl, substituted alkyl, alkoxy, or substituted alkoxy;
R₅ is a hydrogen atom, halogen, hydroxy, alkyl, substituted alkyl, alkoxy, or substituted alkoxy;
either of R₆ or R₇ is —(CH₂)ₙCO₂H or —O(CH₂)ₙCO₂H, wherein n is an integer from 0 to 4, or —(CH₂)ₘ—O—(CH₂)ₚCO₂H, wherein m is an integer from 1 to 2 and p is an integer from 1 to 2, while the other of R₆ or R₇ is a hydrogen atom, halogen, hydroxy, alkyl, substituted alkyl, alkoxy, or substituted alkoxy;
R₈ is a hydrogen atom, halogen, hydroxy, alkyl, substituted alkyl, alkoxy, or substituted alkoxy; and
R₉ is 0-5 phenyl substituents selected from halogen, hydroxy or haloalkyl.

5. The method of claim 4, wherein said neuropathy is hereditary sensory motor neuropathy, diabetic neuropathy, Leber's hereditary optic neuropathy, or Cuban epidemic of optic neuropathy.

6. A method of promoting axonal growth in a human patient comprising identifying a patient in need of such treatment and administering to said patient a therapeutically effective amount of a compound having a structure according to Formula I: 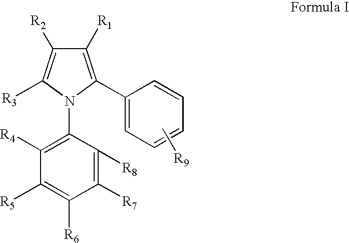
or pharmaceutically acceptable salts thereof, wherein:
R₁ is a hydrogen atom, halogen, hydroxy, alkyl, substituted alkyl, alkoxy, substituted alkoxy, or —CO₂R₁₀, and R₁₀ is alkyl or substituted alkyl;
R₂ is a hydrogen atom, halogen, hydroxy, alkyl, substituted alkyl, alkoxy, substituted alkoxy, or phenyl, optionally substituted with 0-5 phenyl substituents;
R₃ is a hydrogen atom, halogen, hydroxy, alkyl, substituted alkyl, alkoxy, or substituted alkoxy when R₂ is phenyl, or, when R₂ is not phenyl R₃ is —CH₂CH₂-phenyl optionally substituted with 0-5 phenyl substituents;
R₄ is a hydrogen atom, halogen, hydroxy, alkyl, substituted alkyl, alkoxy, or substituted alkoxy;
R₅ is a hydrogen atom, halogen, hydroxy, alkyl, substituted alkyl, alkoxy, or substituted alkoxy;
either of R₆ or R₇ is —(CH₂)ₙCO₂H or —O(CH₂)ₙCO₂H, wherein n is an integer from 0 to 4, or —(CH₂)ₘ—O—(CH₂)ₚCO₂H, wherein m is an integer from 1 to 2 and p is an integer from 1 to 2, while the other of R₆ or R₇ is a hydrogen atom, halogen, hydroxy, alkyl, substituted alkyl, alkoxy, or substituted alkoxy;
R₈ is a hydrogen atom, halogen, hydroxy, alkyl, substituted alkyl, alkoxy, or substituted alkoxy; and
R₉ is 0-5 phenyl substituents selected from halogen, hydroxy or haloalkyl.

7. The method of claim 6, wherein said patient has traumatic brain or spinal cord injury.

8-12. (canceled)

13. The method of claim 2, wherein the compound of Formula I is a compound described in Table 1.

14. The method of claim 2, wherein the compound of Formula I is 4-{4-[2-Methyl-3-phenyl-5-(4-trifluoromethyl-phenyl)-pyrrol-1-yl]-phenyl}-butyric acid.

15. The method of claim 2, wherein the method of treating a neurodegenerative disease comprises delaying the onset of the symptoms of the neurodegenerative disease.

16. The method of claim 2, wherein the method of treating a neurodegenerative disease comprises blocking the onset of the symptoms of the neurodegenerative disease.

17. The method of claim 2, wherein the method of treating a neurodegenerative disease comprises slowing the increase in severity of the symptoms of the neurodegenerative disease.

18-23. (canceled)

24. The method of claim 2, wherein the method of treating a neurodegenerative disease comprises reversing the symptoms of the neurodegenerative disease.

25. The method of claim 4, wherein the compound of Formula I is a compound described in Table 1.

26. The method of claim 4, wherein the method of treating a neuropathy comprises delaying the onset of the symptoms of the neuropathy.

27. The method of claim 4, wherein the method of treating a neuropathy comprises blocking the onset of the symptoms of the neuropathy.

28. The method of claim 4, wherein the method of treating a neuropathy comprises slowing the increase in severity of the symptoms of the neuropathy.

29. The method of claim 4, wherein the method of treating a neuropathy comprises reversing the symptoms of the neuropathy.

30. The method of claim 6, wherein the compound of Formula I is a compound described in Table 1.

31. The method of claim 6, wherein the compound of Formula I is 4-{4-[2-Methyl-3-phenyl-5-(4-trifluoromethyl-phenyl)-pyrrol-1-yl]-phenyl}-butyric acid.

32. The method of claim 6, wherein the compound of Formula I is 4-{4-[2-Methyl-3-phenyl-5-(4-trifluoromethyl-phenyl)-pyrrol-1-yl]-phenyl}-butyric acid.
